(19) United States
(12) Patent Application Publication
(10) Pub. No.: US 2010/0069726 A1
(43) Pub. Date: Mar. 18, 2010

(54) COMPOSITIONS AND METHODS FOR RAPID ONE-STEP DIAGNOSIS

(75) Inventor: Douglas A. Levinson, Sherborn, MA (US)

Correspondence Address: WOLF GREENFIELD & SACKS, P.C., 600 ATLANTIC AVENUE, BOSTON, MA, 02210-2206, US

(73) Assignee: Seventh Sense Biosystems, Inc.

(21) Appl. No.: 12/478,756

(22) Filed: Jun. 4, 2009

Related U.S. Application Data
(60) Provisional application No. 61/058,796, filed on Jun. 4, 2008.
(60) Provisional application No. 61/163,791, filed on Mar. 26, 2009.
(60) Provisional application No. 61/163,793, filed on Mar. 26, 2009.

(51) Int. Cl. A61B 5/00; A61K 49/00

(52) U.S. Cl. 600/309; 424/9.8

(57) ABSTRACT

Devices for fast easy monitoring of analyte levels, disease, or other physiological changes are provided. In some cases, the devices may include particles or the like that can be placed and read at the site of detection, typically on or in the skin or mucosa. In one embodiment, the particles may include anisotropic particles. Typically the devices will typically provide a visual colorimetic signal, but other signals are possible, such as smell, taste (e.g., release of food acceptable flavor), or tactile (e.g., shape change). The devices are preferably single use, disposable devices, although some may be able to provide multiple readings over a period of time. These devices may be used with any patient and are particularly useful for pediatric and elderly patients, as well as for the military, and people without health insurance. The devices can be used to assess when intervention may be required without expensive testing at a physician's office, or simply for routine maintenance of those who are concerned about their health.

### CROSS-REFERENCE TO RELATED APPLICATIONS

This application claims priority to provisional applications U.S. Ser. No. 61/058,796 “Compositions and Methods for Diagnostics, Therapies, and Other Applications”, filed Jun. 4, 2008; U.S. Ser. No. 61/163,791 “Compositions and Methods for Rapid One-Step Diagnosis”, filed Mar. 26, 2009 and U.S. Ser. No. 61/163,793 “Compositions and Methods for Diagnostics, Therapies, and Other Applications”, filed Mar. 26, 2009, all by Douglas Adam Levinson. The disclosures of these applications are incorporated herein by reference.

### FIELD OF INVENTION

In one aspect, the present invention is related to methods and devices for qualitative or quantitative detection of an analyte at the site of detection, typically an intradermal, topical or mucosal site. Another aspect of the present invention is generally directed to a variety of systems and methods generally related to particles, including anisotropic particles having various properties and methods of use thereof.

### BACKGROUND OF THE INVENTION

Many techniques have been developed for the detection and measurements of analytes. Most require removal of the analyte in a fluid or tissue to be measured. Common examples include blood for detection of proteins, cholesterol, or infection. The patient must submit a sample, usually requiring a trained person to collect and process the sample, and then a report is generated, requiring analysis by trained personnel who then must interpret the results for the patient.

Some systems have been developed for the on-line or continuous monitoring of analytes. These range from simple oxygen monitors that clip onto the finger and are hardwired into a monitor that generates a reading of the blood oxygen levels over time, to much more complex monitors that may be inserted into the heart or brain to provide feedback, either hardwired or more recently using wi-fi technology, to a monitor or computer that collects, processes and then reports the results obtained with the monitor. These systems are very complex, and frequently require hospitalization for use.

Simpler outpatient monitoring devices have been developed that provide for more user-friendly output. For example, one can determine blood glucose levels using monitors that require only a single drop of blood, or monitors which are able to extract glucose levels from interstitial fluid. These still require extraction of sample, however. Pregnancy can be determined by application of urine to a strip, which changes color to indicate the presence of human chorionic gonadotropin (hCG), which is secreted by a developing placenta shortly after fertilization.

These devices are all still relatively complex, requiring extraction or removal of sample, and in most cases, reading a level that must be compared with standards for the particular analyte in order to determine if the level is within normal ranges or not. It would reduce the likelihood of user error if one could provide a device that was like the light on an automotive dashboard, saying “low gas” or “needs service” or “battery low”, which was applied at the site where the measurement was obtained. In some cases, this can occur without extraction, without comparison to external values, without calculation, and/or without requiring interpretation by medical personnel.

It is therefore one of many objects of the present invention to provide a device which can provide qualitative, quantitative and/or semi-quantitative analysis of an analyte or condition, at the site of measurement, not requiring external analysis, processing, or comparison to reference values, and methods of use thereof.

### SUMMARY OF THE INVENTION

Devices for fast, easy monitoring of analyte levels, disease states, and/or other physiological changes are provided herein, according to one embodiment. In one embodiment, these function at a basic level which can be analogized to a light on an automotive dashboard—green for normal, yellow for suspicious or cautious, slightly low or slightly high, and red for abnormal. In other embodiments, however, more or fewer signals or levels may be present. The person then knows if he/she needs to be seen, and/or the degree of urgency, by appropriate medical personnel. Such devices may be placed and read at the site of detection, typically on or in the skin or mucosa. Typically the devices will provide a visual colorimetic signal, but other signals are possible, such as smell (released upon change in pH or temperature, for example), taste (bubblegum, cinnamon, or other food acceptable flavor released when device is placed in oral cavity), release of a gas, production of light, electrical or magnetic property, or tactile (shape change due to chemical reaction). In one embodiment, the devices are preferably single use, disposable devices, although some devices may be able to provide multiple readings over a period of time. In other embodiments, the devices may be permanently applied to a subject to be tested. Other uses of devices, e.g., for non-sensing applications, such as cosmetic applications, are also described herein.

Various technologies and reagents useful in certain aspects of the device can be readily used by those of ordinary skill in the art with the benefit of the present disclosure. Additional features such as adhesives, coverings such as bandages, syringes which are preloaded for injection intradermally, can be readily incorporated. For example, devices may be injected into a subject, or the device may be administered to or inserted into the skin of a subject.

As examples, these devices are particularly useful for pediatric, elderly patients, and/or those who suffer from mental illness, who are difficult to test and who are non-compliant, as well as for the military, and people without health insurance (e.g., lower income persons and/or homeless persons). They can be used to assess when intervention may be required without expensive testing at a physician's office, or simply for routine maintenance of those who are concerned about their health.

Thus, a variety of methods generally related to particles, including anisotropic particles having various properties and methods of use thereof, as well as to systems and methods for applying compositions and methods for diagnostics, therapies, and/or other applications, some of which may use such particles and/or other compositions are disclosed herein.

In one preferred embodiment the diagnostic method is a method of determining an analyte. In one set of embodiments, the method includes acts of exposing the analyte to a group of particles, where at least some particles of the group of particles having at least two distinct surface regions including at least a first surface region and a second surface region, and where the first surface region is able to fasten the analyte; fastening the first surface region of the at least some particles to the analyte, thereby forming a plurality of analyte-particle clusters, wherein each cluster includes at least one analyte and first surface regions of particles fastened to the analyte, and where each cluster defines an outer boundary defined by excess of the second surface regions of particles relative to the first surface regions of particles; and determining a determinable feature of the particles, thereby determining the amount or presence of the analyte. In one embodiment, there is a net orientational change in at least one population or subpopulation of particles, e.g., particles oriented on a surface, particles attached to each other, or the like.

In one set of embodiments, the method includes an act of administering a device able to deliver a plurality of skin insertion objects primarily into the epidermis. Preferably the skin insertion objects contain particles, suitable for determining an analyte within the skin of a subject for a period of time of at least about a week following insertion into the skin. In another set of embodiments, the method includes an act of delivering particles, suitable for determining an analyte within the skin of a subject for a period of time of at least about an hour, one day, a week, or longer, to the skin of the subject via a liquid jet process.

In still another set of embodiments, the method is generally directed to an act of administering, into the skin of a subject, particles having at least two distinct regions, each region being present on the surface of the particles. Preferably the method includes an act of determining an analyte in a subject based on the relative positioning of the particles.

In one set of embodiments, the method includes an act of altering coloration of an embedded colorant in a subject by administering an electrical, magnetic, and/or a mechanical force to the subject. The method in still another set of embodiments includes an act of determining an analyte in a subject by determining, in the subject, particles having at least two distinct regions, each region being present on the surface of the particles.

The method, in one set of embodiments, includes the act of providing a subject having skin containing a diagnostic composition suitable for determining an analyte in the subject when applied to the skin of the subject, and applying an externally applied stimulus to the skin of the subject to at least partially remove and/or inactivate the diagnostic composition. In one embodiment, the diagnostic composition contains particles. In a particular embodiment, the particles are removable from the skin. In this embodiment, the method includes the act of applying light to the skin of the subject sufficient to at least partially remove the particles.

The method, according to yet another set of embodiments, includes acts of providing a first particle having at least two distinct regions, each region being present on the surface of the first particle, the first particle containing a first signaling agent; providing a second particle (which in some embodiments may have at least two distinct regions, each region being present on the surface of the second particle), the second particle containing a second signaling agent; and causing the first particle and the second particle to become immobilized relative to each other such that the first signaling agent and the second signaling agent are able to react.

In another set of embodiments, the method includes acts of providing a subject containing administered first and second particles (which in some embodiments may have at least two distinct regions, each region being present on the surface of the particles); and applying a chemical and/or a force to the subject that causes the first particle and the second particle to become immobilized relative to each other. The method, in still another set of embodiments, includes an act of determining a physical condition of a subject by determining the state of a material located in the skin of the subject without applying equipment directly to the subject.

In yet another set of embodiments, the method includes acts of administering, to a subject, first and second particles having at least two distinct regions, each region being present on the surface of the particles; and applying a chemical and/or a force to the subject that causes the first particle and the second particle to become immobilized relative to each other.

Still another embodiment is generally directed to a device for delivery of a plurality of particles to the dermis or epidermis of a subject. According to one set of embodiments, the device contains a substrate; and a plurality of epidermis and/or dermis insertion objects (herein “skin insertion objects), removably fastened to the substrate, optionally carrying a therapeutic, sensory and/or diagnostic agent. In some cases, he substrate is constructed and arranged to apply the plurality of epidermis and/or dermis insertion objects to the skin of a subject and to facilitate introduction of the objects into the epidermis and/or dermis, and is fastened to the plurality of objects at a degree of adhesion such that, when the objects are delivered to the dermis and/or epidermis, at least a portion of the majority of them remain in the dermis and/or epidermis when the substrate is removed from the skin.

Yet another embodiment is generally directed to a diagnostic device. In one set of embodiments, the device contains a plurality of primarily epidermis insertion objects associated with a diagnostic composition, constructed for delivery to the epidermis.

Still another aspect is generally directed to a composition. The composition, in a first set of embodiments, includes a diagnostic composition, suitable for determining an analyte within the epidermis of a subject, dissolved and/or suspended in a fluid suitable for microinjection, microneedle injection, liquid jet delivery, and the like to the epidermis.

Yet another set of embodiments includes a liquid containing first and second particles, the first and second particles each having at least two distinct regions, each region being present on the surface of the particles, where the first particle contains a first signaling agent and the second particle contains a second signaling agent that reacts with the first reactant when the first and second particles are immobilized relative to each other.

Still another aspect is generally directed to a kit for the delivery of a diagnostic or therapeutic agent to the dermis and/or epidermis. The kit, according to one set of embodiments, includes a plurality of skin insertion objects, at least some of which carry a particulate composition comprising a diagnostic or therapeutic agent, constructed and arranged such that, when the plurality of skin insertion objects are applied to the skin, at least some of the particulate composition is delivered to and remains in the dermis and/or epidermis for a diagnostically or therapeutically effective period of time.

In another set of embodiments, the kit includes a first particle having at least two distinct regions, each region being present on the surface of the first particle, the first particle containing a first signaling agent; and a second particle (which in some embodiments may have at least two distinct regions, each region being present on the surface of the second particle), the second particle containing a second signaling agent.

Yet another aspect is generally directed to a cream or a lotion containing a diagnostic composition suitable for determining an analyte associated with a subject when applied to the skin of the subject. Other compositions include those that could be applied to the skin, such as soaps and cosmetics.

Yet another aspect of the invention includes a diagnostic sensor composition foreign to a subject. In some embodiments, the sensor is constructed to be resident in the epidermis of the subject to an extent greater than in the dermis of the subject, where the composition is responsive to an analyte so as to produce a detectable signal in the presence of the analyte distinguishable from a signal in the absence of the analyte. In one aspect, the present invention includes a sensor administrable to the skin of a subject, wherein the sensor determines an analyte using a colorimetric assay.

One aspect includes an article that is an equilibrium-based sensor administrable to a subject. Another aspect includes a homogenous assay administrable to the skin of a subject.

In another aspect, a method of making one or more of the embodiments described herein, for example, an anisotropic particle is provided. In another aspect, a method of using one or more of the embodiments described herein, for example, an anisotropic particle, is provided.

Other advantages and novel features of the devices, compositions, articles, sensors and methods described herein will become apparent from the following detailed description when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

FIGS. 1A-1C illustrate anisotropic particles without an analyte (FIG. 1A), in the presence of an analyte (FIG. 1B), and with four regions (FIG. 1C).

FIGS. 2A-2C illustrate the orientation of anisotropic particles in the presence of an externally applied force (FIGS. 2A and 2C) and in the absence of the externally applied force (FIGS. 2A and 2B).

FIGS. 3A and 3B are a schematic of embodiments of a topical device, shown as placed on the surface of the skin. In FIG. 3B, the topical device contains hollow skin insertion objects.

FIGS. 4A-4C illustrate various skin insertion objects for delivery of particles.

FIGS. 5A-5B illustrate certain techniques for forming anisotropic particles.

FIGS. 6A-6B illustrate anisotropic particles able to react.

### DETAILED DESCRIPTION

Devices for monitoring of analyte levels, disease, or other physiological changes and methods of using the devices are provided. In various embodiments, the devices can be used quickly, easily, and/or by a subject whose condition is being determined. In some cases, the devices include particles or the like that can be placed and read at the site of detection, typically on or in the skin or mucosa. In one embodiment, the particles are anisotropic particles.

The device may contain an assay that can be well controlled, e.g., such that their selectivity, sensitivity, dynamic range, stability, biocompatibility, etc. can be controlled. For instance, a colorimetric assay involving a color change may be controlled by controlling the size of the particles, the colors of the particles, the concentration and/or location of reactive agents on the surfaces of the particles, the anisotropy of the particles, etc. Alternatively, the device may contain a homogeneous assay. Such assays typically do not require any preparation steps, e.g., separation, washing, blocking, etc. In some cases, the assay may be determined without applying any energy and/or external chemicals to the assay, and in some cases, the assay may be determined without the use of any equipment.

### I. Devices

In one embodiment, the diagnostic devices contain at least one reactive agent and signaling agent. In a preferred embodiment, the devices contain one or more particles; in some preferred embodiments the devices contain a plurality of particles. In some embodiments, the devices are in the form of particles. Typically, when the devices are in the form of particles, the particles are administered to a subject in a suitable carrier. In other embodiments, the devices are in a form suitable to administration to a surface of or within the skin or a mucosal surface of a subject without the need for a carrier. Examples of these devices include patches, skin insertion objects, watches, rings, etc. In an embodiment, the device further contains one or more particles, in some embodiments, the particles are anisotropic particles.

Regardless of the form of the device, in a preferred embodiment, the diagnostic device is a single step diagnostic device. As used herein the term “single step diagnostic device” means that in use, the device provides a determinable signal to a user in a single action in addition to the sensing of the result. For example, in some embodiments, the device may be applied on top of or within the skin or mucosal surface of a subject and, after a sufficient period of time, provides a determinable signal, without any additional actions, or steps taken by the user.

However, in some embodiments, the device may be a “two-step” or “multi-step” diagnostic device. For example, in a two-step diagnostic device, a sample may be removed from the subject to be tested (the “first step”), applied to the device (the “second step”) and then, after a sufficient period of time, the device provides determinable signal, without any additional actions, or steps taken by the user.

A. Reactive Agents and Signaling Agents

In certain aspects of the invention, devices such as those described herein may be delivered to a subject, e.g., to the bloodstream or to the skin of a subject, or to a mucosal site within the subject, for various purposes such as for measurement of an analyte, and/or for the delivery of a therapeutic agent, a diagnostic agent, a sensing agent, or in some cases, for cosmetic purposes (e.g., for the creation of a permanent or a temporary tattoo).

For measurement of an analyte, the device includes one or more reactive agents. As used herein, a “reactive agent” or an “analyte reactive agent” means any agent that binds with and/or reacts with analyte to be detected or measured.

A “signaling agent,” as used herein, is an agent that, alone or in combination with another agent, is able to produce a determinable signal. For instance, the signaling agent may be a colored particle, a colorimetric, gold or fluorescent label, a dye, or the like. In some cases, the signaling agent reacts with another agent to produce a determinable signal. For instance, the reaction may produce light, heat, an irritant, or the like, which can be determined, for instance by a subject.

Typically the device contains at least one reactive agent and at least one signaling agent. However, in some embodiments, the reactive agent is also the signaling agent. For example, the device may be a particle, such as an anisotropic particle, and the reactive agent may be an antibody or the like on the surface of the particle. Alternatively, the device may be a patch or contain a substrate that is applied to a mucosal surface on the surface of the skin. In these embodiments, the reactive agent(s) will generally be inside and/or on a surface of the patch or substrate. Other examples of devices and reactive agents are discussed below.

In another embodiment, the device contains more than one reactive agent and more than one signaling agent. This embodiment is particularly useful for determining more than one analyte. For instance, a first set containing at least one reactive agent and at least one signaling agent may determine a first analyte and a second set containing at least one reactive agent that is different from the reactive agents in the first set and at least one signaling agent that is different from the reactive agents in the first set a may determine a second analyte.

A device containing two different antibodies for monitoring the presence and/or amounts of different antigens may also contain two different signaling agents, such as two different colors. For example, a first reactive agent may be an antibody to carcinoembryonic antigen (“CEA”) and a second reactive agent may be an antibody to prostate specific antigen (“PSA”). As a specific non-limiting example, the colors may be yellow for CEA and blue for PSA, resulting in green if both are elevated. In this embodiment, the device may be used to monitor for cancer of either origin, with different colors indicating the presence or likelihood of either or both of the cancers.

Alternatively, the device may contain one reactive agent, which both reacts with the analyte and produces the detectable signal, such as an antibody which is labeled with a signal producing molecule, such as a colorimetric, gold or fluorescent label, which binds the analyte to be detected or measured, and produces a signal that indicates the presence of and/or the amount of analyte. In another embodiment, the signal may be a dye.

The device may be used to determine a physical condition of a subject, such as a healthy level, a potentially dangerous level, or an unhealthy level of a particular analyte. A “subject,” as used herein, includes a human or non-human animal. Examples of subjects include, but are not limited to, a mammal such as a dog, a cat, a horse, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), a bird, a reptile, a fish, or the like.

1. Reactive Agents

The reactive agent binds with and/or reacts with analyte to be detected or measured. As used herein “binding” generally refers to the interaction between a corresponding pair of molecules or surfaces that exhibit mutual affinity or binding capacity, typically due to specific or non-specific binding or interaction, including, but not limited to, biochemical, physiological, and/or chemical interactions. The binding may be between biological molecules, including proteins, nucleic acids, glycoproteins, carbohydrates, and/or hormones. Specific non-limiting examples of molecules that bind to each other include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc.

Reactive agents may bind specifically, semi-specifically, or even non-specifically to the analyte of interest. In the preferred embodiment, the reactive agent binds specifically or semi-specifically with the analyte to be measured or detected, more preferably specifically. However, in other embodiments, reactive agents that have other interactions with the analyte of interest, including non-specific interactions, may be used.

As used herein “specifically binds,” when referring to a reactive agent that binds to an analyte to be detected or measured, refers to a reaction that is determinative of the presence and/or identity of analyte in a mixture of heterogeneous molecules (e.g., proteins and other biologics). Thus, for example, in the case of a receptor/ligand binding pair, the ligand specifically and/or preferentially binds to its receptor from a complex mixture of molecules, or vice versa. An enzyme specifically binds to its substrate; a nucleic acid specifically binds to its complement; an antibody specifically binds to its antigen, etc.

The binding may be by one or more of a variety of mechanisms including, but not limited to ionic interactions or electrostatic interactions, covalent interactions, hydrophobic interactions, van der Waals interactions, hydrogen bonding, etc.

In one embodiment, the reactive agent that binds with and/or reacts with the analyte to be detected or measured may to form specific, non-covalent, physiochemical interactions with the analyte.

Many reactive agents that specifically bind with analytes are known in the art, and include any molecular species, including, but not limited to antibodies, which bind to antigen, ligands that bind to receptors, enzymes that bind to substrates and nucleic acids that bind complementary nucleic acids, and aptamers, i.e. oligonucleic acid or peptide molecules that bind a specific target molecule, chelating agents, and ion selective polymers. In some cases, binding may be between non-biological molecules, for example, between a catalyst (e.g., the reactive agent) and its substrate. The reactive agent may be biotin, which binds to streptavidin as the analyte to be detected or measured, or vice versa. Alternatively, the reactive agent may be various antibodies raised against a protein to be detected or measured.

Various non-limiting examples of reactive agents that may be included in the device are described below.

a. Chelating Agents

The reactive agent may be a chelating agent. Suitable chelating agents in ethylenediamine tetraacetic acid (EDTA); diethylenetriamine pentaacetic acid (DTPA); N-(hydroxyethyl)ethylenediaminetriacetic acid (HEDTA); nitrilotriacetic acid (NTA); histidine; malate; phytochelatins, such as oligomers of glutathione, homophytochelatin, desglycine phytochelatin, hydroxymethyl-phytochelatin, and iso-phytochelatin; porphyrin rings, such as hemoglobin and chlorophyll; water-soluble pigments that act as chelating agents, such as siderophores; citric acid; phosphonates; tetracyclines; polycarboxylic acid polymers, such as acrylic acid polymers and copolymers; ascorbic acid; tetrasodium iminodisuccinate; dicarboxymethylglutamic acid; ethylenediaminedisuccinic acid (EDDS); hepta sodium salt of diethylene triamine penta (methylene phosphonic acid) (DTPMP•Na7); hydrolysed wool; nitrilotriacetic acid (NTA); nonpolar amino acids, such as methionine; oxalic acid; phosphoric acid; polar amino acids, such as arginine, asparagine, aspartic acid, glutamic acid, glutamine, lysine, and ornithine; succinic acid; dimercaprol; and combinations thereof.

b. Ion Selective Polymers

The reactive agent may be an ion selective polymer. Suitable ion selective polymers include, but are not limited to, block copolymers such as poly(carbonate-b-dimethylsiloxane); crown ethers, thiacrown ethers, azacrown ethers, or immobilized derivatives thereof where the crown ether is immobilized on a polymer; polytetrtafluoroethylene, to which charged groups (e.g., cationic, anionic, and/or zwitterionic groups); and polyols immobilized on a substrate, such as a polymer, and functionalized with charged groups, such as ethylene glycol, glycerol, tris(hydroxymethyl)ethane, pentaerythritol, and pentaerythritol triethoxylate immobilized onto a polymer, such as cross-linked poly(vinylbenzyl chloride), and phosphorylated.

The ion selective polymer can be a molecularly imprinted ion-selective polymer, such as those described Molecularly Imprinted Polymers by Börje Sellergen, Elsevier Science BV, The Netherlands (2001) and discussed in more detail below. In embodiments where the analyte to be detected has an intrinsic chromophore or other means of detection, in some cases a requirement is binding affinity and stability (i.e., stable for the time period required for measurement). Alternatively, the polymer can be responsible for the signal (e.g., optical signal) that is detected. In these embodiments, binding of the analyte can occur at a site that influences the atom or groups of atoms that is responsible for producing the signal to be detected. For example, ligands for metals (or other analytes) can be chosen that increase the analyte's molar absorptivity or yield a colored complex. Examples include Pb²⁺ and dithizone. For metal ions (or other analytes) that do not exhibit color, the analyte can be coordinated by ligands that form a fluorescent complexes, such as Zn²⁺ with benzoin. As described below, a second reagent can be added which reacts with the Zn²⁺/benzoin complex to produce a species which emits in the visible region of the spectrum. In cases where the analyte is negatively charged, luminescent metal ions can be chosen as a component of the binding site to acquire both a thermodynamic binding affinity and a suitable chromophore.

c. Antibodies

The reactive agent may be an antibody that binds to a particular epitope in the antigen of interest. Typical epitopes include, but are not limited to, hemagglutin (HA), FLAG® (Sigma-Aldrich), c-Myc, glutatione-S-transferase, His₆, green fluorescent protein (GFP), digoxigenin (DIG), biotin or avidin. Antibodies that bind to these epitopes are well known in the art. Antibodies may be monoclonal or polyclonal.

Suitable antibodies for use as reactive agents that bind to an analyte to be detected include, but are not limited to, antigen-binding fragments of one or more antibodies, including separate heavy chains, light chains Fab, Fab' F(ab')₂, Fabc, and Fv. Antibodies also include bispecific or bifunctional antibodies. Exemplary binding partners of a reactive agent and its corresponding analyte include biotin/avidin, biotin/streptavidin, biotinlneutravidin and glutathione-S-transferase/glutathione.

For example, Protein A is a reactive agent, which may be used to bind to the biological molecule IgG, and vice versa. Protein A is usually regarded as a “non-specific” or semi-specific binder. An enzyme such as glucose oxidase or glucose 1-dehydrogenase, or a lectin such as concanavalin A that is able to bind to glucose, may also be utilized in the devices described herein.

Other non-limiting examples of suitable reactive agents include nucleic acids that bind complementary nucleic acids, nucleic acids that bind proteins, proteins that bind other proteins, enzymes that bind substrate, receptors that bind ligand, receptors that bind hormones and antibodies that bind antigen.

2. Signaling Agents

The signaling agent generates a signal that can be determined in some fashion. In some embodiments, more than one signaling agent may be required to produce the determinable signal. “Determine,” in this context, generally refers to the analysis of a species, for example, quantitatively or qualitatively, and/or the presence or absence of the species. “Determining” may also refer to the analysis of an interaction between two or more species, for example, quantitatively or qualitatively, and/or the presence or absence of the interaction, e.g. determination of the binding between two species. As an example, an analyte may cause directly or indirectly a determinable change in a property of the device or at least one of the signaling agents present in the device, e.g., a change in a chemical property, appearance and/or optical properties, temperature, and/or an electrical property. Generally, the change is determinable by a human, unaided by any equipment that may be directly applied to or used by a human with the exception of devices ordinarily used by the individual, such as glasses or a hearing aid. For instance, the determinable change may be a change in appearance (e.g., color), a change in temperature, the production of an odor, etc., which can be determined by a human without the use of any additional equipment.

In one embodiment, the one or more signaling agents are on the outer surfaces of one or more particles, typically anisotropic particles. In one embodiment the particles are in surface of an object, typically a diagnostic device, or a substrate or a film. Preferably, the particles are able to orient so that they bind to the surface of the object.

a. pH Sensitive Reagents

One example of a signaling agent is a pH-sensitive reagent. Exemplary pH-sensitive reagents include, but are not limited to, phenol red, bromothymol blue, chlorophenol red, fluorescein, HPTS (8-Hydroxypyrene-1,3,6-trisulfonic acid, trisodium salt, 5(6)-carboxy-2′,7′-dimethoxyfluorescein SNARF® (Molecular Probes, Invitrogen), and phenothalein.

b. Reagents Sensitive to the Presence of Ions or Molecules

In another embodiment, the signaling agent may be a reagent that is sensitive to the presence of an ion, such as a cation, an anion, or both, or a molecule, such as O₂, CO₂, NH₃, fatty acids, proteins, glucose, etc.

Examples include, but are not limited to, reagents sensitive to calcium such as Dura-2 and Indo-1; entities sensitive to chloride such as 6-methoxy-N-(3-sulfopropyl)-quinolinim and lucigenin; entities sensitive to nitric oxide such as 4-amino-5-methylamino-2′,7′-difluorofluorescein; entities sensitive to dissolved oxygen such as tris(4,4′-diphenyl-2,2′-bipyridine) ruthenium (II) chloride pentahydrate; entities sensitive to dissolved CO₂; entities sensitive to fatty acids, such as BODIPY 530-labeled glycerophosphoethanolamine; entities sensitive to proteins such as 4-amino-4′-benzamidostilbene-2-2′-disulfonic acid (sensitive to serum albumin), X-Gal or NBT/BCIP (sensitive to certain enzymes), Tb³⁺ from TbCl₃ (sensitive to certain calcium-binding proteins), BODIPY FL phallacidin (sensitive to actin), or BOCILLIN FL (sensitive to certain penicillin-binding proteins); entities sensitive to concentration of glucose, lactose or other components, or entities sensitive to proteases, lactates or other metabolic byproducts, entities sensitive to proteins, antibodies, or other cellular products.

Other properties of the signaling agent besides, or in addition to, color may be determined in other embodiments, e.g., temperature changes, and/or chemical reactions (e.g., produced by capsaicin). For example, in one embodiment, the signaling agent contains capsaicin or capsaicin-like molecules. Examples of capsaicin and capsaicin-like molecules, which may be used as the signaling agent include, but are not limited to, dihydrocapsaicin, nordihydrocapsaicin, homodihydrocapsaicin, homocapsaicin, or nonivamide. A signal produced by capsaicin or a capsaicin-like molecule may be felt or sensed by a subject as a change in temperature or a burning sensation (due to reaction with sensory neurons), although the mechanism of the capsaicin reaction does not necessarily include an actual temperature change.

c. Color Signals

The signaling agent and/or the devices may be colored or react or reorient within a surface of the subject or the surface of the device to produce a change or an appearance of a change in color. For instance, in one embodiment, the signaling agent may be a particle or a portion or region within a particle, such as anisotropic particle, that exhibits a change in visual appearance, e.g., a change in overall color, hue, shading, texture (e.g., from uniform to non-uniform or “patchy” or a heterogeneous appearance), reflective versus non-reflective, etc. when exposed to an analyte. The color, hue, shading, texture (e.g. uniform color versus clumpy appearance or heterogenous mixture of colors), reflectivity (e.g. from reflective to non-reflective) changes, and/or the intensity of the particular color, may vary. In one embodiment, the signaling agent may produce or release color or another indicator, hydrolyse or release a particular color when reacted, or aggregate to intensify a color when reacted.

For example, one or more signaling agents may produce a first color, which indicates a healthy state and the same or different reactive agent(s) may produce a second color, which indicate a disease state. In some cases, the appearance of the device, such as the particular color, may be used to indicate the patient's degree of health with respect to one or more analytes. For instance, a first color may indicate a healthy state, a second color may indicate a warning state, and a third color may indicate a dangerous state, or a range of colors may indicate a degree of health of the subject.

For example, anisotropic particles containing two or more regions, may contain a reactive agent in the first region and a signaling agent in the first or the second region.

As a specific example, the first set of particles containing two regions may be colored yellow in the first region and blue in the second region, and the second set of particles containing two regions may be colored red in the first region and blue in the second region. If no analyte is present, the reactive agents are randomly oriented, giving a dark appearance (i.e., red+yellow+blue). A different reactive agent may be present in either region of each set of particles. In one embodiment, the first set of particles contains a first reactive agent in the second region, which is colored blue, and, optionally, the second set of particles contains a second reactive agent, which binds to or interacts with the same or a different analyte than the first reactive agent, in the second region, colored blue. If both sets of particles contain the same reactive agent, but at different concentrations within the particles, then they may be used to determine relative amounts of concentration of the analyte that is present. For example, if the analyte is present, but at low concentrations, the first set of reactive agents may be able to bind the analyte but not the second set of reactive agents, as the first set of reactive agents contain a higher concentration of reactive agents able to recognize the analyte. Thus the first set of reactive agents may exhibit more yellow than blue (e.g., due to aggregation of the first set of reactive agents to the analyte; the first set of reactive agents may aggregate around the analyte to a greater degree than the second set of reactive agents), and the overall appearance of the reactive agents shifts to a dark yellow appearance. At higher concentrations of analyte, both sets of reactive agents may be able to bind the analyte, and the second set of reactive agents may exhibit more red than blue (e.g., due to aggregation of the second set of reactive agents). The overall appearance of the reactive agents may then shift to an orange appearance (red+yellow).

In one embodiment, the reactive agent may be labeled with a signaling agent. In this embodiment, the reactive agent behaves as the signaling agent. For example, if the reactive agent is an antibody, the antibody may be fluorescently labeled. Thus, when the antibody reacts with the analyte to be detected, it fluoresces producing a determinable signal.

Alternatively, an optical property of a medium containing the devices may be altered in some fashion (e.g., exhibiting different light scattering properties, different opacities, different degrees of transparency, etc.), which can be correlated with the analyte. In some cases, the color may change in intensity, for example, the clustering of particles may bring two or more signaling agents into close proximity.

In another embodiment, the device may contain two signaling agents. For example, when the reactive agent binds with the analyte, the first signaling agent produces a signal that is not easily detected, for example, fluorescence in the UV region of the spectrum. The second signaling agent reacts with the complex of the analyte and first reactive agent to produce a signal that is more easily observed, for example, emission in the visible region of the spectrum (i.e., colored species).

d. Other Properties

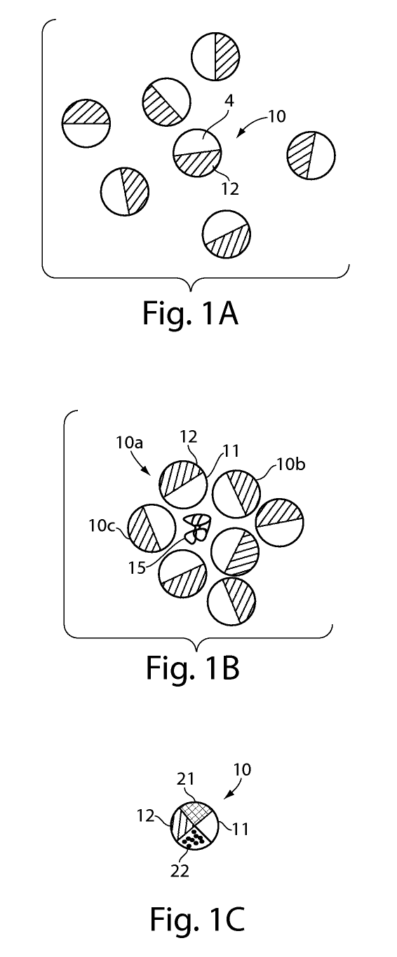

Other properties may also be determined besides color. Accordingly, it should be understood that the use of “color” as used herein is by way of example only, and other properties may be determined instead of or in addition to color. For instance, clustering of aniostropic particles may cause a change in an electrical or a magnetic property of the particles, which can be determined by determining an electrical or a magnetic field. For example, a plurality of particles 10 surrounding an analyte 15, as illustrated in FIG. 1B, may produce particles having a different magnetic moment than isolated particles, which can be determined by determining a magnetic property of the particles.

As another example, the first region and the second region of the particles may have different reactivities (e.g., the first region may be reactive to an enyzme, an antibody, etc.), and aggregation of the particles may cause a net change in the reactivity, which can be determined.

As still another example, size may be used to determine the particles and/or the analyte. For instance, the aggregates may be visually identifiable, the aggregates may form a precipitant, or the like. Thus, for example, the particles (which may be anisotropic or not anisotropic) may appear to be a first color when separated, and a second color when aggregated.

In some cases, an assay (e.g., an agglutination assay) may be used to determine the state of the particles, i.e. whether aggregation has occurred.

In another set of embodiments, an ordering of the particles may be determined. For example, in the absence of an analyte, the particles may be ordered on the surface of a substrate; while in the presence of an analyte, the particles may bind to the analyte and become disordered relative to the surface. This ordering may be determined, for example, as a change in an optical property of the surface (e.g., index of refraction, color, opacity, etc.).

As yet other examples, a shape change may be produced using a shape memory polymer or a “smart polymer. Examples of these are discussed below.

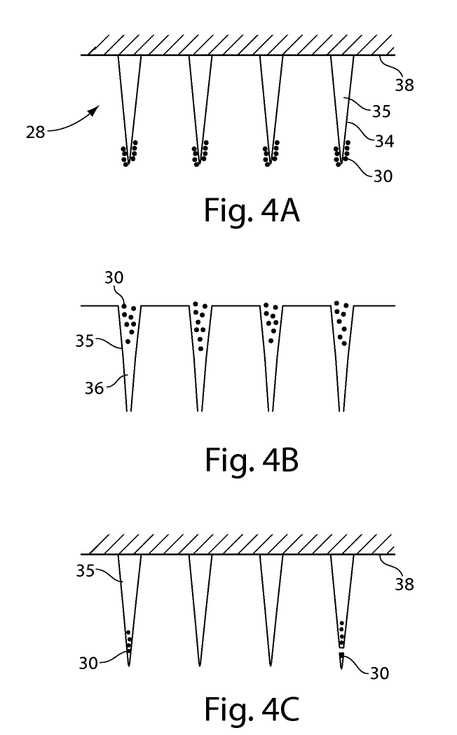

The clustering or aggregation of particles as discussed herein is not limited to generally spherical aggregations. In some cases, the particles may cluster onto a surface, or the particles may be aligned in some fashion relative to the surface due to an analyte or other external force. In FIG. 4B, the particles may be aligned, for example, by an externally applied magnetic field, which may be reversible in some cases.

The Signaling Agent May be Detected in any Fashion

The signaling agents may react in any fashion that can be determined, either directly, or by determining a property of the devices that contain the signaling agents, such as by producing light, emitting or absorbing heat (i.e. increase or decrease in temperature), pH change, release of a gas, smell, taste, texture, compound that produces a sensation (e.g., irritation or pain), etc. In some cases, a precipitate and/or flocculate may be formed—or may disperse. In another example, clustering of signaling agents and/or devices that contain the signaling agents may cause a change in an electrical or a magnetic property of the signaling agents and/or devices that contain the signaling agents, which can be indicative of a change in an electrical or a magnetic field. As a specific example, particles, such as anisotropic particles, may contain one or more signaling agents that produce light, emit heat, etc., upon exposure to an analyte.

In some cases, the aggregates may precipitate and/or flocculate. For instance, if particles are present in a solution, the particles may form aggregates that may separate from the solution, and optionally can be removed or otherwise analyzed. As yet another example, an aggregate of particles may form in the absence of analyte, but disaggregate (at least partially) in the presence of the analyte, e.g., if the analyte and the particles exhibit competitive or non-competitive inhibition. Such binding and/or aggregation may be equilibrium-based in some cases, i.e., the binding and/or aggregation occurs in equilibrium with unbinding or disaggregation processes. Thus, when the environment surrounding the particles is altered in some fashion (e.g., a change in concentration of an analyte), the equilibrium may shift in response, which can be readily determined (e.g., as a change in color). It should be noted that such equilibrium-based systems may be able to determine such changes in environment, in some cases, without the need to apply any energy to determine the environmental change.

Temperature Change

The reaction between a first and a second signaling agent may be an endothermic or an exothermic reaction; resulting in a detectable temperature change. As an example, the device may contain a reactive agent and as a first signaling agent, barium hydroxide (Ba(OH)₂), and as a second signaling agent, ammonium nitrate (NH₄NO₃). In one embodiment, the device contains a plurality of particles, which may be anisotropic or non-anisotropic. In this embodiment, the first signaling agent may be on a first set of particles, and the second signaling agent may be on a second set of particles. However, in another embodiment, the particles may contain two or more regions, where the first signaling agent is in a first region of the particle, and the second signaling agent is in a different region than the first signaling agent on the same particle. The signaling agents may be present in solution or suspension, and only a low level of reaction between the barium hydroxide and the ammonium nitrate occurs. However, when a species is added which is recognized by the reactive agent, aggregation of the particles may occur. As the particles aggregate to orient on the species, the first and second signaling agents may also be brought into closer proximity, allowing the reaction rate between the signaling agents to increase. In this case, the reaction between barium hydroxide and the ammonium nitrate is an endothermic reaction that yields barium nitrate (Ba(NO₃)₂) and ammonium (NH₃). This may be determined by determining a drop in temperature.

The devices may also contain as a reactive agent a glucose reactive agent, such as a lectin (e.g., concanavalin A), glucose oxidase or glucose 1-dehydrogenase, that is able to bind to glucose. At relatively low levels of glucose, little or no aggregation of the devices occurs, and no change in temperature is felt by the subject. However, at relatively high levels of glucose, some aggregation of the devices occurs, such that the devices orient around the glucose, bringing the reactive agents into close proximity to each other, allowing the reaction rate between the reactive agents to increase. In this case, the reaction between barium hydroxide and the ammonium nitrate is an endothermic reaction that yields barium nitrate (Ba(NO₃)₂) and ammonium (NH₃). This may be sensed as a drop in temperature.

Irritation or Pain

Irritation or pain can also be used as the signal that is detected. As an example, a device may release an irritant upon interaction of a reactive agent with a species that to which the reactive agent binds or interacts. For example, a glucose sensor can be prepared from devices formed of a biocompatible polymer such as PEO, or a polymer of polylactic acid and/or polyglycolic acid. The first set of devices contains a reactive agent to a species and the first signaling agent, while the second set of devices also contains a reactive agent to the species (which may be the same or different than the reactive agent of the first set of devices) and a second signaling agent. The first and second signaling agents may be, for example, two agents that cause the release of capsaicin or a capsaicin-like molecule such as dihydrocapsaicin, which may be felt by a subject as pain. In one embodiment, the first device may be a liposome that contains the capsaicin or capsaicin-like molecule and the second device may be a lipase able to degrade the liposome, thereby releasing the capsaicin from the liposome. The first set of devices also contains as the reactive agent, a glucose reactive agent, such as a lectin (e.g., concanavalin A), glucose oxidase or glucose 1-dehydrogenase that is able to bind to glucose. In another embodiment, the devices may contain particles, such as anisotropic particles.

e. Tactile Changes

Shape Memory Polymers

In another embodiment, the binding or presence of the analyte results in a tactile change (e.g., change in shape or texture) in the composition. For example, shape memory polymer (SMPs) or “smart polymers” can be used as signaling agents to detect the presence of one or more analytes.

In the literature, SMPs are generally characterized as phase segregated linear block co-polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature. The melting point or glass transition temperature of the soft segment is substantially less than the melting point or glass transition temperature of the hard segment.

When the SMP is heated above the melting point or glass transition temperature of the hard segment, the material can be shaped. This (original) shape can be memorized by cooling the SMP below the melting point or glass transition temperature of the hard segment. When the shaped SMP is cooled below the melting point or glass transition temperature of the soft segment while the shape is deformed, that (temporary) shape is fixed. The original shape is recovered by heating the material above the melting point or glass transition temperature of the soft segment but below the melting point or glass transition temperature of the hard segment. The recovery of the original shape, which is induced by an increase in temperature, is called the thermal shape memory effect. Properties that describe the shape memory capabilities of a material are the shape recovery of the original shape and the shape fixity of the temporary shape.

Shape memory polymers can contain at least one physical crosslink (physical interaction of the hard segment) or contain covalent crosslinks instead of a hard segment. The shape memory polymers also can be interpenetrating networks or semi-interpenetrating networks. In addition to changes in state from a solid to liquid state (melting point or glass transition temperature), hard and soft segments may undergo solid to solid state transitions, and can undergo ionic interactions involving polyelectrolyte segments or supramolecular effects based on highly organized hydrogen bonds.

Other polymers that can change shape or phase as a function of temperature include PLURONICS®. These are also known as poloxamers, nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the PLURONICS® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In the example given, poloxamer 181 (P181)=Pluronic L61. PLURONICS® are described in U.S. Pat. No. 3,740,421.

Other temperature sensitive polymers that form gels that have a distinct phase change at its lower critical solution temperature (LCST) including the cross-linked copolymers comprising hydrophobic monomers, hydrogen bonding monomers, and thermosensitive monomers described in U.S. Pat. No. 6,538,089 to Samra, et al.

Additional thermal responsive, water soluble polymers including the co-polymerization product of N-isopropyl acrylamide (NIP); 1-vinyl-2-pyrrolidinone (VPD); and optionally, acrylic acid (AA), change shape as a function of temperature. As the proportion of component AA increases, the Lower Critical Solution Temperature (LCST) decreases and the COOH reactive groups increase, which impart high reactivity to the copolymer. By adjusting the proportion of the monomers, a broad range of LCST can be manipulated from about 20 to 80° C., as described in U.S. Pat. No. 6,765,081 to Lin, et al.

While the shape memory effect is typically described in the context of a thermal effect, the polymers can change their shape in response to application of light, changes in ionic concentration and/or pH, electric field, magnetic field or ultrasound. For example, a SMP can include at least one hard segment and at least one soft segment, wherein at least two of the segments, preferably two soft segments, are linked to each other via a functional group that is cleavable under application of light, electric field, magnetic field or ultrasound. The temporary shape is fixed by crosslinking the linear polymers. By cleaving those links the original shape can be recovered. The stimuli for crosslinking and cleaving these bonds can be the same or different.

In one embodiment, the shape memory polymer composition binds, complexes to, or interacts with an analyte, which is a chromophore. The hard and/or soft segments can include double bonds that shift from cis to trans isomers when the chromophores absorb light. Light can therefore be used to detect the presence of a chromophore analyte by observing whether or not the double bond isomerizes.

The shape memory effect can also be induced by changes in ionic strength or pH. Various functional groups are known to crosslink in the presence of certain ions or in response to changes in pH. For example, calcium ions are known to crosslink amine and alcohol groups, i.e., the amine groups on alginate can be crosslinked with calcium ions. Also, carboxylate and amine groups become charged species at certain pHs. When these species are charged, they can crosslink with ions of the opposite charge. The presence of groups, which respond to changes in the concentration of an ionic species and/or to changes in pH, on the hard and/or soft segments results in reversible linkages between these segments. One can fix the shape of an object while crosslinking the segments. After the shape has been deformed, alteration of the ionic concentration or pH can result in cleavage of the ionic interactions which formed the crosslinks between the segments, thereby relieving the strain caused by the deformation and thus returning the object to its original shape. Because ionic bonds are made and broken in this process, it can only be performed once. The bonds, however, can be re-formed by altering the ionic concentration and/or pH, so the process can be repeated as desired. Thus, in this embodiment, the presence of an analyte which changes the ionic strength or pH can induce a shape memory effect in the polymer confirming the presence of the analyte.

Electric and/or magnetic fields can also be used to induce a shape memory effect. Various moieties, such as chromophores with a large number of delocalized electrons, increase in temperature in response to pulses of applied electric or magnetic fields as a result of the increased electron flow caused by the fields. After the materials increase in temperature, they can undergo temperature induced shape memory in the same manner as if the materials were heated directly. These compositions are particularly useful in biomedical applications where the direct application of heat to an implanted material may be difficult, but the application of an applied magnetic or electric field would only affect those molecules with the chromophore, and not heat the surrounding tissue. For example, the presence of a chromophore analyte with a large number of delocalized electrons can be cause an increase in temperature in the microenvironment surrounding the shape memory polymer implant in response to pulses of applied electric or magnetic fields. This increase in temperature can in turn cause a thermal shape memory effect, thus confirming the presence of a particular analyte.

Many other types of “smart polymers” are described in U.S. Pat. No. 5,998,588 to Hoffman, et al. The combination of the capabilities of stimuli-responsive components such as polymers and interactive molecules to form site-specific conjugates are useful in a variety of assays, separations, processing, and other uses. The polymer chain conformation and volume can be manipulated through alteration in pH, temperature, light, or other stimuli. The interactive molecules can be biomolecules like proteins or peptides, such as antibodies, receptors, or enzymes, polysaccharides or glycoproteins which specifically bind to ligands, or nucleic acids such as antisense, ribozymes, and aptamers, or ligands for organic or inorganic molecules in the environment or manufacturing processes. The stimuli-responsive polymers are coupled to recognition biomolecules at a specific site so that the polymer can be manipulated by stimulation to alter ligand-biomolecule binding at an adjacent binding site, for example, the biotin binding site of streptavidin, the antigen-binding site of an antibody or the active, substrate-binding site of an enzyme. Binding may be completely blocked (i.e., the conjugate acts as an on-off switch) or partially blocked (i.e., the conjugate acts as a rheostat to partially block binding or to block binding only of larger ligands). Once a ligand is bound, it may also be ejected from the binding site by stimulating one (or more) conjugated polymers to cause ejection of the ligand and whatever is attached to it. Alternatively, selective partitioning, phase separation or precipitation of the polymer-conjugated biomolecule can be achieved through exposure of the stimulus-responsive component to an appropriate environmental stimulus.

Liquid crystal polymeric materials can also be used to provide a signal for detection or quantitation of analyte. Liquid crystals are materials that exhibit long-range order in only one or two dimensions, not all three. A distinguishing characteristic of the liquid crystalline state is the tendency of the molecules, or mesogens, to point along a common axis, known as the director. This feature is in contrast to materials where the molecules are in the liquid or amorphous phase, which have no intrinsic order, and molecules in the solid state, which are highly ordered and have little translational freedom. The characteristic orientational order of the liquid crystal state falls between the crystalline and liquid phases. Suitable materials are described in U.S. Pat. Nos. 6,465,002 and 6,696,075 by Mathiowitz, et al. These can be pressure or temperature sensitive, and react by producing a change in color or shape.

f. Other Interactions Between Two or More Signaling Agents

In addition, it should be noted that more than one signaling agent may be required to produce a determinable signal. For instance, there may be a first set of particles containing a first signaling agent and a second signaling agent that reacts with the first signaling agent. When the particles are brought together in some fashion (e.g., by exposure to an analyte or other chemical that is recognized by reactive agents on each of the particles, by the application of an electrical, magnetic, and/or a mechanical force to bring the particles closer together, etc.), the first and second signaling agents may react with each other.

As a specific example, the reaction between the first and second signaling agents may be an endothermic or an exothermic reaction; thus, when the particles are brought together, a temperature change is produced, which can be determined in some fashion.

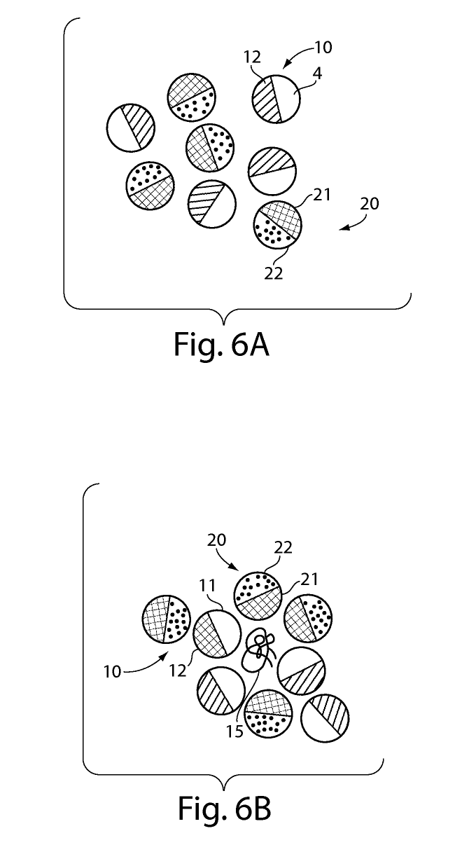

For example, as is shown in FIG. 6A, a first particle 10 having a first region 11 containing a first reactive agent that binds to or interacts with an analyte and a second region 12 containing a first signaling agent may be brought together with a second particle 20 having a first region 21 containing a second reactive agent that binds to or interacts with an analyte and a second region 22 containing a second signaling agent In FIG. 6B, an analyte 15 is introduced, which brings particles 10 and 20 together, accordingly bringing regions 22 and 12 into close proximity. If these signaling agents are reactive with each other, by providing an analyte, a reaction between the first and second signaling agents can be induced or at least accelerated by brining the reactive agents closer together. The first and second signaling agents may be any suitable agents that react with each other to produce a determinable signal. For instance, the first and second reactive agents can produce heat (e.g., as in an exothermic reaction), cold (e.g., as in an endothermic reaction), a change in color, a product which can then be determined, or the like.

As another example, a reaction between the first and second signaling agents may cause the release of a material. In some cases, the material may be one that can be sensed by a subject, e.g., capsaicin, an acid, an allergen, or the like. Thus, the subject may sense the change as a change in temperature, pain, itchiness, swelling, or the like. Other examples include agents that cause vasodilation or vasoconstriction, histamine, irritants (e.g., capsaicin, venoms, such as venoms from bees, scorpions, fire ants, etc), colorants, dyes, effervescent agents, agents that produce an odor upon release, etc.

Reaction between the first and second reactive agents may cause the release of one or more therapeutics, diagnostic, and/or prophylactic agents. Exemplary classes of therapeutic agents include, but are not limited to, analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants (anorexic agents); attention deficit disorder and attention deficit hyperactivity disorder drugs; cardiovascular agents including calcium channel blockers, antianginal agents, central nervous system (“CNS”) agents, beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; sialagogues, steroids; smoking cessation agents; sympathomimetics; tranquilizers; vasodilators; beta-agonist; and tocolytic agents.

Exemplary therapeutic agents include, but are not limited to, ceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolarn, clozapine, codeine, corticosterone, cortisone, cyclobenzaprine, cyproheptadine, dapoxetine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

B. Particles.

In some embodiments, the device contains one or more particles and preferably contains a plurality of particles. In another embodiment, the particles are diagnostic devices themselves. For example, anisotropic particles can be utilized as analyte detection devices.

The particles can be used in a wide variety of applications. For example, the particles may include a reactive agent that when exposed to an analyte recognized by the reactive agent, causes the particles to collect around the analyte, e.g., as an aggregate, as previously discussed. The aggregate may produce a visual or other signal distinguishable from the particles in a non-aggregated state, such as a randomly-oriented state. In some cases, the particles, when aggregated, may allow a chemical reaction to occur, which produces a detectable signal.

a. Microparticles and Nanoparticles

The particles may be microparticles and/or nanoparticles. A “microparticle” is a particle having an average diameter on the order of micrometers (i.e., between about 1 micrometer and about 1 mm), while a “nanopartiele” is a particle having an average diameter on the order of nanometers (i.e., between about 1 nm and about 1 micrometer). In some cases, a plurality of particles may be used, and in some cases, some, or substantially all, of the particles may be the same. For example, at least about 5%, at least about 10%, at least about 30%, at least about 40%, at least about 50%, at least about 60%, at least about 70%, at least about 80%, at least about 90%, at least about 95%, or at least about 99% of the particles may have the same shape, and/or may have the same composition. For example, in one embodiment, at least about 50% of the particles may be anisotropic.

b. Anisotropic Particles

In one set of embodiments, particles used in the subject to determine the analyte are anisotropic particles (in other cases, however, the particles are not necessarily anisotropic), and in some cases, substantially all of the particles are anisotropic particles. In certain cases, at least about 10%, at least about 30%, at least about 40%, at least about 50%, at least about 60%, at least about 70%, at least about 80%, at least about 90%, at least about 95%, or at least about 99% of the particles are anisotropic particles. In one embodiment, the anisotropic particles may have a first region having a first color and a second region having a second color distinct from the first color, and the particles, upon exposure to the analyte within the subject, may form clusters that exhibit an excess of the second region or second color relative to the first region or first color, as discussed above. The particles may be present, for example, in the bloodstream, interstitial fluid, and/or within the skin of the subject (e.g., temporary tattoo within the epidermis). If the particles are delivered to the skin of the subject, the particles may be delivered to any location within the skin (or below the skin), e.g., to the epidermis, to the dermis, subcutaneously, intramuscularly, etc. In some cases, a “depot” of particles may be formed within the skin, and the depot may be temporary or permanent. For instance, the particles within the depot may eventually degrade (e.g., if the particles are biodegradable), enter the bloodstream, or be sloughed off to the environment. As an example, if the particles are delivered primarily to the epidermis, many of the particles can eventually be sloughed off to the environment (as the epidermis is sloughed off), i.e., such that the particles are present within the subject on a temporary basis (e.g., on a time scale of days or weeks). However, if the particles are delivered to lower layers of tissue, e.g., to the dermis or lower, then the particles may not be as readily sloughed off to the environment (or the particles may take longer to be sloughed off into the environment), and thus the particles may be present in the skin on a longer basis. For instance, the particles may be present within the subject for weeks, months, or years.

An “anisotropic” particle, as used herein, is one that is not spherically symmetric (although the particle may still exhibit various symmetries). The asymmetry can be asymmetry of shape, of composition, or both. As an example, a particle having the shape of an egg or an American football is not perfectly spherical, and thus exhibits anisotropy. As another example, a sphere painted such that exactly one half is red and one half is blue (or otherwise presents different surface characteristics on different sides) is also anisotropic, as it is not perfectly spherically symmetric, although it would still exhibit at least one axis of symmetry. Accordingly, a particle may be anisotropic due to its shape and/or due to two or more regions that are present on the surface of and/or within the particle. The particle may include a first surface region and a second surface region that is distinct from the first region in some way, e.g., due to coloration, surface coating, the presence of one or more reactive agents, etc. The particle may include different regions only on its surface or the particle may internally include two or more different regions, portions of which extend to the surface of the particle. The regions may have the same or different shapes, and be distributed in any pattern on the surface of the particle. For instance, the regions may divide the particle into two hemispheres, such that each hemisphere has the same shape and/or the same surface area, or the regions may be distributed in more complex arrangements. For instance, a first region may have the shape of a circle on the surface of the particle while the second region occupies the remaining surface of the particle, the first region may be present as a series of distinct regions or “spots” surrounded by the second region, the first and second regions may each be present as a series of “stripes” on the surface of the particle, etc. In some cases, the particle may include three, four, five, or more distinct surface regions. For instance, a particle may include distinct first, second and third surface regions; distinct first, second, third, and fourth surface regions; distinct first, second, third, fourth and fifth surface regions, etc. In some cases, the surface regions may be distinctly colored, and in certain instances, the anisotropic particles may be able to exhibit multiple colors, depending on the external environment. For example, a particle may exhibit a first color in response to a first analyte and a second color in response to a second analyte, as discussed below.

In the absence of analyte, the anisotropic particles may be oriented randomly, as is illustrated in FIG. 1A, with particles (10) containing a first region (11) and a second region (12). In the presence of analyte (15), however, some of the particles (10a, b and c) may orient towards the analyte, and in some cases may surround the analyte (see FIG. 1B). Thus, the analyte can alter the orientation of the particles.

Interactions between the particle and the analyte can be competitive. In one embodiment, analyte competes with binding between the particles in a concentration dependent manner. The greater the concentration of analyte, the less binding occurs between the particles, and the greater the signal. In contrast, low analyte concentration results in greater particle-particle binding and thus less signal. In another embodiment, binding between the analyte and the reactive agent results in one signal and binding between particles results in a different signal. At high concentrations of analyte, binding is primarily between analyte and reactive agent, while at low concentrations, binding is primarily between particles.

If, for instance, a reactive agent is present in the first region (11) of the particles but not the second region (12), the color in the second region (12) may dominate the first color as the particles orient towards the analyte (15), as shown in FIG. 1B. Accordingly, by exposing the analyte to such anisotropic particles, a plurality of analyte-particle clusters may form, and in some embodiments, the clusters may exhibit an excess of the second surface region relative to the first surface region of the particles.

FIG. 1C illustrates anisotropic particles that are able to exhibit a first color in response to a first analyte and a second color in response to a second analyte. In FIG. 1C, particle 10 contains a first region (11) a second region (12), a third region (21), and a fourth region (22). The first region (11) may contain a reactive agent that binds to a first analyte, while third region (21) may contain a second reactive agent that binds to a second analyte. Thus, in the presence of the first analyte, the particle may present second region (12) (e.g., a first color), while in the presence of the second analyte, the particle may present fourth region (22) (e.g., a second color). Thus, the particles may be used to determine the presence and/or relative amounts of two different analytes.

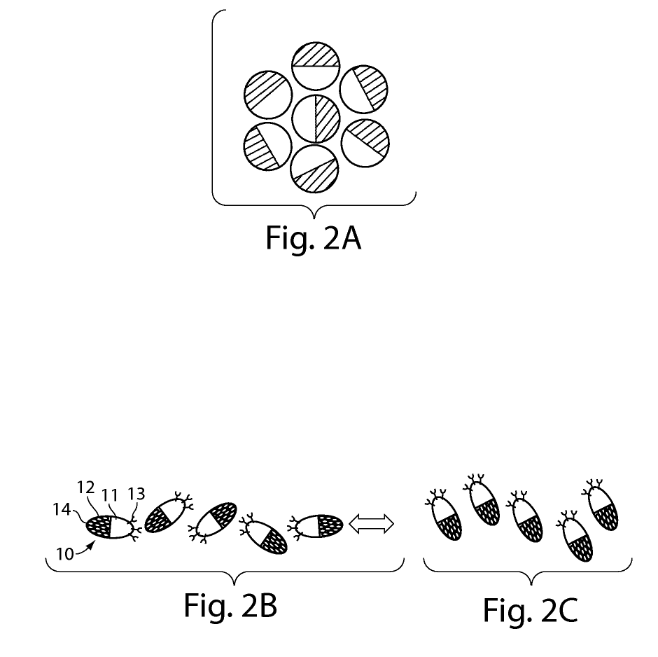

In another embodiment, the application of an electrical, magnetic, and/or a mechanical force to the particles causes the particles to exhibit a change in color. For example, if at least a portion of the particles are magnetically permeable, the application of a magnetic field may cause the particles to form clusters. This can be seen in FIG. 2A, where randomly distributed particles, such as shown in FIG. 1A, are induced to form particle clusters as shown in FIG. 2A under the influence of an externally applied magnetic field.

As shown in FIG. 2B, anisotropic particles (10) containing a first region (11) and a second region (12), may be controlled by an external force, such as an externally applied magnetic field. In this example, the first region (11) contains a reactive agent (13), and the second region (12) may contain, for example, another agent (14), such as a therapeutic agent, a sensory agent, or a color (e.g., produced by a dye, a colorimetric agent, a fluorescent entity, a phosphorescent entity, etc.).

Non-limiting examples of anisotropic particles are disclosed in U.S. patent application Ser. No. 11/272,194, filed Nov. 10, 2005, entitled “Multi-phasic Nanoparticles,” by J. Lahann, et al., published as U.S. Publication No. 2006/0201390 on Sep. 14, 2006; U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Publication No. 2007/0237800 on Oct. 11, 2007; or U.S. Provisional Patent Application Ser. No. 61/058,796, filed Jun. 4, 2008, entitled “Compositions and Methods for Diagnostics, Therapies, and Other Applications,” by Douglas A. Levinson, each of which is incorporated herein by reference.

U.S. Publication No. 2003/0159615 by Anderson, et al., describes a wide variety of microparticles containing and/or formed of colored dyes, which can be used to create a colored signal.

c. Materials

The particles (which may be anisotropic, or not anisotropic) may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these. In one set of embodiments, the particles may comprise a hydrogel, such as agarose, collagen, or fibrin.

d. Magnetically Susceptible Material

The particles may include a magnetically susceptible material in some cases, e.g., a material displaying paramagnetism or ferromagnetism. For instance, the particles may include iron, iron oxide, magnetite, hematite, or some other compound containing iron. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles include ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs.

e. Additional Agents

The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa). In one embodiment, in addition to containing one or more reactive agents and/or one or more signaling agents, the particles also contains one or more therapeutic agents to treat the disease or disorder that is identified using the reactive agents.

Exemplary classes of therapeutic agents include, but are not limited to, analeptic agents; analgesic agents; anesthetic agents; antiasthmatic agents; antiarthritic agents; anticancer agents; anticholinergic agents; anticonvulsant agents; antidepressant agents; antidiabetic agents; antidiarrheal agents; antiemetic agents; antihelminthic agents; antihistamines; antihyperlipidemic agents; antihypertensive agents; anti-infective agents; anti-inflammatory agents; antimigraine agents; antineoplastic agents; antiparkinsonism drugs; antipruritic agents; antipsychotic agents; antipyretic agents; antispasmodic agents; antitubercular agents; antiulcer agents; antiviral agents; anxiolytic agents; appetite suppressants (anorexic agents); attention deficit disorder and attention deficit hyperactivity disorder drugs; cardiovascular agents including calcium channel blockers, antianginal agents, central nervous system (“CNS”) agents, beta-blockers and antiarrhythmic agents; central nervous system stimulants; diuretics; genetic materials; hormonolytics; hypnotics; hypoglycemic agents; immunosuppressive agents; muscle relaxants; narcotic antagonists; nicotine; nutritional agents; parasympatholytics; peptide drugs; psychostimulants; sedatives; sialagogues, steroids; smoking cessation agents; sympathomimetics; tranquilizers; vasodilators; beta-agonist; and tocolytic agents.

Reaction between the first and second reactive agents may cause the release of one or more therapeutics, diagnostic, and/or prophylactic agents. Exemplary therapeutic agents include, but are not limited to, ceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, dapoxetine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pen azocine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In another embodiment, the particles can be those that, based on their degree or amount of dispersion or agglomeration, produce a different signal. For example, certain particles or colloids such as gold nanoparticles can be coated with agents capable of interacting with an analyte. Such particles may associate with each other, or conversely, dissociate in the presence of analyte in such a manner that a change is conferred upon the light absorption property of the material containing the particles. For example, particles coated with complimentary nucleic acid sequences can be used to characterize target nucleic acids complimentary to the particle bound nucleic acids sequence. This approach can also be applied to any class of analyte, in various embodiments, and furthermore can be used as a skin-based visual sensor. A non-limiting example of a technique for identifying aggregates is disclosed in U.S. Pat. No. 6,361,944.

f. Sizes and Shapes

The particles may have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm.

The particles may be spherical or non-spherical. For example, the particles may be oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al., published as U.S. Publication No. 2008/0112886 on May 15, 2008; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Publication No. 2007/0237800 on Oct. 11, 2007, each of which is incorporated herein by reference. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks.

g. Particles as Diagnostic Devices

In another embodiment, the particles are diagnostic devices themselves. In this embodiment, the particles may be administered to a subject using a suitable carrier. For example, in one embodiment, the particles are administered via injection. The particles can be administered as solution, suspension, or emulsion. Suitable carriers for injection of the particles include, but are not limited, to sterile saline, phosphate buffered saline, water, ethanol, polyol (for example, glycerol, propylene glycol, and liquid polyethylene glycol, and the like), suitable mixtures thereof, and oil, such as vegetable oils. The formulation may contain one or more pharmaceutically acceptable excipients, such as dispersants, pH modifying agents, buffering agents, surfactants, isotonic agents, preservatives, water soluble polymers (e.g., polyethylene glycols, polyvinyl pyrrolidone, dextran, and carboxymethyl cellulose), and combinations thereof.

In another embodiment, the particles may be administered topically to the surface of a subject's skin or mucosal surface using a suitable carrier. Suitable carriers for topical administration of the particles include gels, foams, ointments, pastes, and lotions. The cream or lotion may contain, for instance, an emulsion of a hydrophobic and a hydrophilic material (e.g., oil and water), distributed in any order (e.g., oil-in-water or water-in-oil), and the particles may be present in any one or more of the emulsion phases.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Alternatively, the particles may be mucoadhesive and may be sprayed onto the mucosal surface of the tissue. For example, the particles may be formed from mucoadhesive polymers. Mucoadhesive polymers can be classified in two groups: hydrogels and hydrophilic polymers. Mucoadhesive polymers typically contain functional groups that adhere to tissue, such as carboxylic acid groups, hydroxyl groups, and/or amine groups. Classes of mucoadhesive polymers include, but are not limited to, poly vinylpyrrolidone (PVP), methyl cellulose (MC), sodium carboxy methylcellulose (SCMC) hydroxy propyl cellulose (HPC) and other cellulose derivatives, Carbopol, polyacrylates and crosslinked polyacrylates, chitosan and derivatives thereof (N-trimethyl chitosan), acrylic resins, available under the tradename Eudragits®, poly(dimethyl-aminoethyl methacylate) (PDMAEMA), and combinations thereof.

Kits

In one embodiment, an apparatus may be used to deliver the particles to a subject. For instance, the apparatus may be a syringe or vial. The apparatus may be included in a kit. For example, the kit may contain a syringe, containing lyophilized or dried microparticles and a suspending agent such as sterile saline or phosphate buffered saline in a kit.

h. Particles as a Component in a Diagnostic Device

In some embodiments, the device contains one or more particles and preferably contains a plurality of particles. This embodiment is described in more detail below.

C. Forms for Devices

a. Particles

In one embodiment, described above, the devices are in the form of particles. In one embodiment, the particles are in a form suitable for injection. Alternatively, the particles may be designed for topical application to the surface of the skin or a mucosal surface. In each of these embodiments, the particles are administered using a suitable carrier.

b. Non-Injectable Devices

In another embodiment, the device is non-injectible embodiment. In one embodiment the device is applied to the skin or a mucosal surface (mouth, sublingual, rectal, vaginal). The device include at a minimum two components: (1) a display monitor, surface, or signal release feature and (2) an analyte receiving or reaction chamber or surface. The two components may be contiguous or even a single dual purpose component. The device also contains one or more reactive agents and one or more signaling agents. In one embodiment, the signaling agents are designed to align with the outer surface of the device to produce a determinable signal.

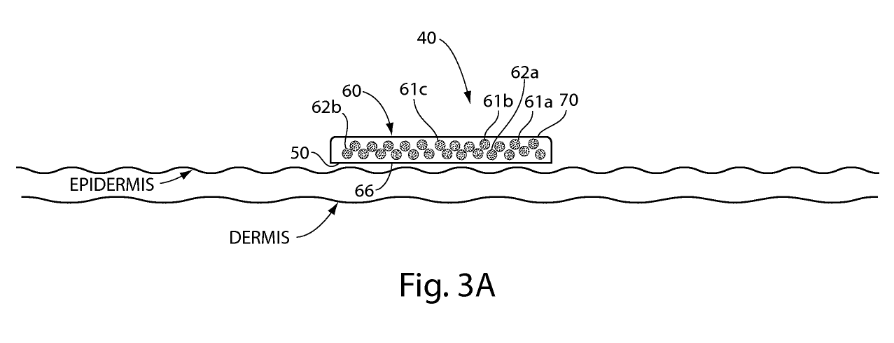

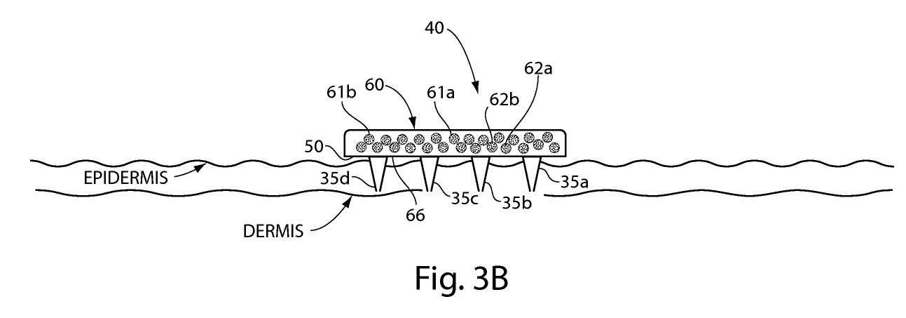

An exemplary device for placement on the skin or a mucosal surface is provided in FIG. 3. As shown in FIG. 3, the device (40) typically contains a substrate layer (50) and a chamber (60), optionally, the device also contains an outer layer (70).

In one embodiment, the device contains a substrate layer (50) formed of a biocompatible material that is suitable for applying to the surface of the user. In one embodiment, this layer is adhesive. Skin adhesives range in degree and length of duration, and can be obtained commercially. For example, they may be cyanoacrylates for long term wound closure, or lightly adhesive of the type found on wound coverings such as BANDAID®s, or a UV-inpenetrable transparent skin patch.

The chamber (60) contains one or more reactive agents (62a, b, c) and one or more signaling agents (61a and b). Typically, the side (66) of the chamber that is proximal to the surface of the user is permeable, at least, to the analyte to be detected. This allows for analyte transfer from the user into the device.

In some embodiments the outer layer (70) is impermeable to gases, while in other embodiments it allows for gas exchange. For example, in those embodiments in which the detectable signal is a scent emitted by the reactive agent(s), the device preferably includes a gas permeable outer layer to allow the user to smell the scent.

In one embodiment, the device contains hollow or solid skin insertion objects. An example of this embodiment is illustrated in FIG. 3B, in which, the skin insertion objects (35a, b, c and d) are attached to the substrate layer (50) via the side (66) of the chamber that is proximal to the surface of the user. Optionally the skin insertion objects are hollow and are designed to allow for the transfer of bodily fluids, such as blood or interstitial fluid, from the body into the substrate to contact the reactive agent(s).

The device may be in the form of a ring, bracelet, watch, earrings, or other devices which are physically restrained at the site of contact. Generally, in these embodiments, the device will not contain an adhesive layer proximal to the skin surface since the device is typically applied to the surface using alternative means, such as a physical restraint.

The devices may be applied by application of an adhesive or physical restraint. Skin adhesives range in degree and length of duration, and can be obtained from 3M, Johnson & Johnson, and a variety of other medical supply companies. These may be cyanoacrylates for long term wound closure, or lightly adhesive of the type found on wound coverings such as BANDAID®s. A uv-inpenetrable transparent skin patch is described in U.S. Pat. No. 5,811,108 to Goeringer, which can be utilized in making a suitable transdermal device.

Mucosal Devices

The device may be applied to a patient's oral cavity and more specifically, the lingual and sub-lingual regions of the oral cavity. The underside and base of the tongue, as well as the base of the oral cavity beneath the tongue, are highly variegated and vascularized, containing capillaries close to the surface, which presents a considerable surface area to allow for transfer of analyte for detection and measurement.

The device may be in the form of a film, patch or other adhesive that adheres to the sublingual space, trapping the analyte in or on the device. Alternatively a powdered composition containing micro- or nano-particles may be delivered to the oral cavity, such as to the upper surface of the tongue, and more preferably to the sublingual space.

a. Mucoadhesive Patches or Bandages

The device may adhere to mucosal surfaces and dissolve or otherwise disintegrate over time, delivering particles into mucosal surface in a sustained fashion. The device may contain at least one surface with a composition that exhibits good adherence to human oral mucosa. The device may be formed of a bioadhesive material or have one or more surfaces coated or formed of a bioadhesive material which adheres to a mucosal surface in the oral cavity, vaginal or rectal areas.

In some embodiments, the particles may contain a mucoadhesive material. In some cases, the particles may be sprayed onto the tissue, e.g., when the reaction is detected by a color change.

Buccal tablets are known. See, for example, in U.S. Pat. Nos. 4,740,365 and 4,764,378.

Adhesives for use with non-mucosal adhesive devices that adhere to mucosal surfaces are known to the art. Polyacrylic acids and polyisobutylenes have been disclosed as components of such adhesives. For example, U.S. Pat. No. 3,339,546 to Chen discloses a bandage that is said to adhere to moist surfaces of the oral cavity and comprises a medicament and a hydrocolloid (carboxypolymethylene (i.e., polyacrylic acid)) incorporated in a natural or synthetic gum-like substance. U.S. Pat. No. 4,615,697 to Robinson discloses a composition including a bioadhesive and a treating agent. The bioadhesive is a water-swellable but water insoluble, fibrous, crosslinked, carboxy-functional polymer containing a plurality of repeating units of which at least about 80% contain at least 1 carboxy functionality, and about 0.05 to about 1.5% of a cross-linking agent substantially free from polyalkenyl polyether. U.S. Pat. No. 4,253,460 to Chen et al. discloses an adhesive composition consisting of a mixture of a hydrocolloid gum, a pressure sensitive adhesive, and a cohesive strengthening agent. The pressure sensitive adhesive component can be a mixture of three to five parts of a polyisobutylene with a viscosity average molecular weight of about 36,000 to about 53,000 and one part of an elastomer such as a polyisobutylene with a viscosity average molecular weight of about 1,150,000 to about 1,600,000. U.S. Pat. No. 4,740,365 to Yukimatsu et al. discloses a sustained-release preparation comprising an active ingredient and a mixture of two polymer components, the first of which comprises polyacrylic acid or a pharmaceutically acceptable salt thereof, and the second is polyvinylpyrrolidone, polyvinyl alcohol, polyethylene glycol, alginic acid, or a pharmaceutically acceptable salt of alginic acid. CARBOPOL® resins are among the polymers said to be suitable members of the first-mentioned class of polymers. U.S. Pat. No. 4,772,470 to Inoue, et al. discloses an oral bandage comprising a mixture of a polyacrylic acid and a vinyl acetate polymer in a compatible state. This bandage is said to exhibit strong adhesion of long duration when applied to oral mucosa or teeth.

Mucoadhesive polymers are defined as polymers that have an adherence to living mucosal tissue of at least about 110 N/m² of contact area (11 mN/cm²). A suitable measurement method is set forth in U.S. Pat. No. 6,235,313 to Mathiowitz et al. Polyanhydrides are a preferred type of mucoadheisve polymer. The mechanism causing the anhydride polymers or oligomers to be bioadhesive is believed to be due to a combination of the polymer's hydrophobic backbone, coupled with the presence of carboxyl groups at the ends. Interaction of charged carboxylate groups with tissue has been demonstrated with other bioadhesives. In particular, pharmaceutical industry materials considered to be bioadhesive typically are hydrophilic polymers containing carboxylic acid groups, and often hydroxyl groups as well. The industry standard is often considered to be CARBOPOL™ (a high molecular weight poly(acrylic acid)). Other classes of bioadhesive polymers are characterized by having moderate to high densities of carboxyl substitution. The relatively hydrophobic anhydride polymers frequently demonstrate superior bioadhesive properties when compared with the hydrophilic carboxylate polymers.

Suitable polyanhydrides include polyadipic anhydride, poly fumaric anhydride, polysebacic anhydride, polymaleic anhydride, poly malic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different molar ratios.

Natural adhesives for underwater attachment of mussels, other bivalves and algae to rocks and other substrates are known (see U.S. Pat. No. 5,574,134 to Waite, U.S. Pat. No. 5,015,677 to Benedict et al., and U.S. Pat. No. 5,520,727 to Vreeland et al.). These adhesives are polymers containing poly(hydroxy-substituted) aromatic groups. In mussels and other bivalves, such polymers include dihydroxy-substituted aromatic groups, such as proteins containing 3,4-dihydroxyphenylalanine (DOPA). In algae, diverse polyhydroxy aromatics such as phloroglucinol and tannins are used. In adhering to an underwater surface, the bivalves secrete a preformed protein that adheres to the substrate thereby linking the bivalve to the substrate. After an initial adherence step, the natural polymers are typically permanently crosslinked by oxidation of adjacent hydroxyl groups. The attachment of DOPA to different polymeric backbones is described in U.S. Pat. No. 4,908,404 to Benedict et al. and U.S. Publication No. 2005/0201974 to Schestopol et al. Suitable mucoadhesive polymers include DOPA-maleic anhydride co polymer; isopthalic anhydride polymer; DOPA-methacrylate polymers; and DOPA-cellulosic based polymers.

Bioadhesive materials contain a polymer with a catechol functionality. The molecular weight of the bioadhesive materials and percent substitution of the polymer with the aromatic compound may vary greatly. The degree of substitution varies based on the desired adhesive strength, it may be as low as 10%, 20%, 25%, 50%, or up to 100% substitution. On average at least 50% of the monomers in the polymeric backbone are substituted with at least one aromatic group. Preferably, 75-95% of the monomers in the backbone are substituted with at least one aromatic group or a side chain containing an aromatic group. In the preferred embodiment, on average 100% of the monomers in the polymeric backbone are substituted with at least one aromatic group or a side chain containing an aromatic group. The resulting bioadhesive material is a polymer with a molecular weight ranging from about 1 to 2,000 kDa. The polymer that forms that backbone of the bioadhesive material may be any non-biodegradable or biodegradable polymer. In the preferred embodiment, the polymer is a hydrophobic polymer. In one embodiment, the polymer is a biodegradable polymer and is used to form an oral dosage formulation.

Examples of preferred biodegradable polymers include synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers.

Mucoadhesive materials also include poly(fumaric acid:sebacic acid), as described in U.S. Pat. No. 5,955,096 to Mathiowitz et al., incorporating oligomers and metal oxides polymer to enhance the ability of the polymer to adhere to a tissue surface such as a mucosal membrane, as described in U.S. Pat. No. 5,985,312 to Jacob et al. Preferably, the polymer is a biodegradable polymer.

D. Additional Agents or Materials

a. Analyte Transfer Enhancers

Prior to or simultaneous with administering the devices on or into the skin or a mucosal surface, one or more chemical enhancers may be administered to the site of administration of the device. Chemical enhancers have been found to increase transdermal drug transport via several different mechanisms, including increased solubility of the drug in the donor formulation, increased partitioning into the SC, fluidization of the lipid bilayers, and disruption of the intracellular proteins (Kost and Langer, In Topical Drug Bioavailability, Bioequivalence, and Penetration; Shah and Maibech, ed. (Plennum, NY 1993) pp. 91-103 (1993)). See also U.S. Pat. No. 5,445,611 to Eppstein, et al.

Lipid Bilayer Disrupting Agents.

Chemical enhancers have been found to increase drug transport by different mechanisms. Chemicals which enhance permeability through lipids are known and commercially available. For example, ethanol has been found to increase the solubility of drugs up to 10,000-fold (Mitragotri, et al. In Encl. of Pharm. Tech.: Swarbrick and Boylan, eds. Marcel Dekker 1995) and yield a 140-fold flux increase of estradiol, while unsaturated fatty acids have been shown to increase the fluidity of lipid bilayers (Bronaugh and Maiback, editors (Marcel Dekker 1989) pp. 1-12).

Examples of fatty acids which disrupt lipid bilayer include linoleic acid, capric acid, lauric acid, and neodecanoic acid, which can be in a solvent such as ethanol or propylene glycol. Evaluation of published permeation data utilizing lipid bilayer disrupting agents agrees very well with the observation of a size dependence of permeation enhancement for lipophilic compounds. The permeation enhancement of three bilayer disrupting compounds, capric acid, lauric acid, and neodecanoic acid, in propylene glycol was reported by Aungst, et al. Pharm. Res. 7, 712-718 (1990).

A comprehensive list of lipid bilayer disrupting agents is described in European Patent Application 43,738 (1982). Exemplary compounds are represented by the formula:

R—X

wherein R is a straight-chain alkyl of about 7 to 16 carbon atoms, a non-terminal alkenyl of about 7 to 22 carbon atoms, or a branched-chain alkyl of from about 13 to 22 carbon atoms, and X is —OH, —COOCH₃, —COOC₂H₅, —OCOCH₃, —SOCH₃, —P(CH₃)₂O, COOC₂H₄OC₂H₄OH, —COOCH(CHOH)₄CH₂OH, —COOCH₂CHOHCH₃, COOCH₂CH(OR″)CH₂OR″, —(OCH₂CH₂)ₘOH, —COOR′, or —CONR₂ where R′ is —H, —CH₃, —C₂H₅, —C₂H₇ or —C₂H₄OH; R″ is —H, or a non-terminal alkenyl of about 7 to 22 carbon atoms; and in is 2-6; provided that when R″ is an alkenyl and X is —OH or —COOH, at least one double bond is in the cis-configuration.

Solubility Enhancers

Suitable solvents include water; dials, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl)pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

U.S. Pat. No. 4,537,776 to Cooper contains a summary of prior art and background information detailing the use of certain binary systems for permeant enhancement. European Patent Application 43,738, also describes the use of selected dials as solvents along with a broad category of cell-envelope disordering compounds for delivery of lipophilic pharmacologically-active compounds. A binary system for enhancing metaclopramide penetration is disclosed in UK Patent Application GB 2,153,223 A, consisting of a monovalent alcohol ester of a C8-32 aliphatic monocarboxylic acid (unsaturated and/or branched if C18-32) or a C6-24 aliphatic monoalcohol (unsaturated and/or branched if C14-24) and an N-cyclic compound such as 2-pyrrolidone or N-methylpyrrolidone.

Combinations of enhancers consisting of diethylene glycol monoethyl or monomethyl ether with propylene glycol monolaurate and methyl laurate are disclosed in U.S. Pat. No. 4,973,468 for enhancing the transdermal delivery of steroids such as progestogens and estrogens. A dual enhancer consisting of glycerol monolaurate and ethanol for the transdermal delivery of drugs is described in U.S. Pat. No. 4,820,720. U.S. Pat. No. 5,006,342 lists numerous enhancers for transdermal drug administration consisting of fatty acid esters or fatty alcohol ethers of C₂ to C₄ alkanediols, where each fatty acid/alcohol portion of the ester/ether is of about 8 to 22 carbon atoms. U.S. Pat. No. 4,863,970 discloses penetration-enhancing compositions for topical application including an active permeant contained in a penetration-enhancing vehicle containing specified amounts of one or more cell-envelope disordering compounds such as oleic acid, oleyl alcohol, and glycerol esters of oleic acid; a C₂ or C₃ alkanol and an inert diluent such as water.

Other chemical enhancers, not necessarily associated with binary systems, include dimethylsulfoxide (DMSO) or aqueous solutions of DMSO such as those described in U.S. Pat. No. 3,551,554 to Herschler; U.S. Pat. No. 3,711,602 to Herschler; and U.S. Pat. No. 3,711,606 to Herschler, and the azones (n-substituted-alkyl-azacycloalkyl-2-ones) such as noted in U.S. Pat. No. 4,557,943 to Cooper.

Some chemical enhancer systems may possess negative side effects such as toxicity and skin irritations. U.S. Pat. No. 4,855,298 discloses compositions for reducing skin irritation caused by chemical enhancer-containing compositions having skin irritation properties with an amount of glycerin sufficient to provide an anti-irritating effect.

Combinations of Lipid Bilayer Disrupting Agents and Solvents

In some embodiments, lipid bilayer disrupting agents and solvents may be administered to the same site, prior to or simultaneous with the administration of the device. Ultrasound with polyethylene glycol 200 dilaurate (PEG), isopropyl myristate (IM), and glycerol trioleate (GT) results in corticosterone flux enhancement values of only 2, 5, and 0.8, relative to the passive flux from PBS alone. However, 50% ethanol and LA/ethanol significantly increase corticosterone passive fluxes by factors of 46 and 900, indicating that the beneficial effects of chemical enhancers and therapeutic ultrasound can be effectively combined. Ultrasound combined with 50% ethanol produces a 2-fold increase in corticosterone transport above the passive case, but increase by 14-fold the transport from LA/Ethanol.

b. Mechanical, Electrical and Ultrasound Transducers

Ultrasound, mechanical abrasion and/or electrical fields can be used to enhance transdermal transfer of the analyte through the skin or the mucosal surface. Echo Therapeutics, Franklin, Mass. has a SonoPrep® system that includes ultrasound-based skin permeation technology for a non-invasive and painless method of enhancing the flow of molecules across the skin's membrane for up to 24 hours. The SonoPrep system and its method of use are described in a variety of U.S. Patents, including U.S. Pat. Nos. 6,190,315; 6,234,990; 6,491,657; 6,620,123.

Echo's application of ultrasonic energy creates reversible channels in the skin through which large molecules can be delivered or removed for analysis. This use of ultrasound technology makes it possible for painless and transdermal drug delivery or analyte extraction. The SonoPrep® system operates by transferring a low level of ultrasound energy for a short time from the hand piece, causing the outer most layer of skin (stratum corneum) to become permeable. The size of the sonication site is typically 0.8 cm². Echo has conducted studies to demonstrate that skin conductivity is significantly enhanced and that the enhancement lasts for several hours. The SonoPrep® system provides real-time skin conductance feedback. SonoPrep® measures the increase in skin conductance (or decrease in skin impedance) during the application of ultrasound and stops the sonocation procedure when the desired level of conductance has been achieved. This technology can be incorporated into the methods and compositions described herein to provide rapid easy one-step monitoring.

c. Monitors

Monitors can be embedded into a non-injectable device, such as a bandage or a reservoir type device having an area containing color changing chromophores, LEDs, liquid crystal display, or other materials may be incorporated into the device itself. Liquid crystals, as described above, can be bioerodible or non-bioerodible. Representative non-mesogenic, bioerodible polymers include polylactic acid, polylactide-co-glycolide, polycaprolactones, polyvaleric acid, polyorthoesters, polysaccharides, polypeptides, and certain polyesters. Representative mesogenic, bioerodible polymers include some polyanhydrides and polybutylene terephthalate. Preferred non-mesogenic, non-erodible polymers include polyethylene, polypropylene, polystyrene, and polytherephthalic acid. The polymer can be water-soluble or water-insoluble. These can be used in the controlled release or retention of substances encapsulated in the LC polymers. The polymer can be in a variety of forms including films, film laminants, and microparticles. In a preferred embodiment, the LC polymers are used to encapsulate therapeutic, diagnostic, or prophylactic agents for use in medical or pharmaceutical applications. Other substances which can be encapsulated include scents such as perfumes, flavoring or coloring agents, sunscreen, and pesticides.

The LC polymer can be made in a variety of forms including films, film laminants, coatings, membranes, microparticles, slabs, extruded forms, and molded forms. The LC polymers can be combined with each other, with non-LC polymers, or with other materials such as metals, ceramics, glasses, or semiconductors, the latter typically in the form of coatings. The polymers can be fabricated into articles and then treated to induce the LC state, or the LC state can be induced and then articles formed from the LC polymer. Compositions that include the LC polymers can be monolithic or layered. The term “monolithic” is used herein to describe a continuous phase having imbedded structures, rather than layers. The LC polymers can be prepared separately and then mixed with other materials in a process that does not change the transition temperature. LC polymers can be used in display systems, such as for computers, and in message systems wherein a message can be displayed or hidden from view based on changes in the opacity/transparence of the LC polymer which occur with changes in the crystal structure of the material. LC polymers also can be used in product packaging. Another application for the LC polymers is in temperature sensing devices, for example. In one medical application, the sensor is attached to the skin to provide a temperature map indicating local temperature variations. Such devices are useful, for example, in the diagnosis of certain medical ailments, such as tumors, or areas of infection or inflammation or poor circulation which have a temperature different from the surrounding healthy tissue.

The monitor can be a switchable responsive device administered with or incorporated within the particles. The switch can be detected by adding another detector, which is able to detect the switch (e.g., an LED in a bandage that shines light on a mark).

### II. Methods of Manufacture

A. Particles

Microparticles and nanoparticles can be prepared using a variety of techniques known in the art. The functional groups used to bind or complex the analyte can be introduced prior to microparticle formation (e.g., monomers can be functionalized with one or more functional groups for binding or complexing the analyte) or the functional groups can be introduced after microparticle formation (e.g., by functionalizing the surface of the microparticle with reactive functional groups). The microparticles may optionally have encapsulated therein one or more core materials. In one embodiment, the microparticles or nanoparticles should be present in an effective amount to provide a signal detectable to the user without the need for additional equipment. For example, the microparticles and/or nanoparticles should be present in an effective amount to provide a change in taste, smell, shape, and/or color upon binding or complexing the analyte that is easily detectable by the user.

The following are representative methods for forming microparticles and nanoparticles. Techniques other than those described below may also be used to prepare microparticles and/or nanoparticles.

Anisotrophic Microparticles

Techniques for forming anisotrophic particles or fibers can be found in U.S. patent application Ser. No. 11/272,194, filed Nov. 10, 2005, entitled “Multi-Phasic Nanoparticles,” by Lahann, et al., published as U.S. Patent Application Publication No. 2006/0201390 on Sep. 14, 2006; or priority to U.S. patent application Ser. No. 11/763,842, filed Jun. 15, 2007, entitled “Multiphasic Biofunctional Nano-Components and Methods for Use Thereof,” by Lahann, published as U.S. Patent Application Publication No. 2007/0237800 on Oct. 11, 2007.

Solvent Evaporation

In solvent evaporation the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting nanoparticles and microparticles are washed with water and dried overnight in a lyophilizer. Particles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

Solvent Removal

Solvent removal techniques are primarily designed for polyanhydrides. In this method, the polymer is dissolved in a volatile organic solvent like methylene chloride. The mixture is suspended by stirring in an organic oil (such as silicon oil) to form an emulsion. Unlike solvent evaporation, this method can be used to make nanoparticles from polymers with high melting points and different molecular weights. Nanoparticles that range between 1-300 microns can be obtained by this procedure. The external morphology of spheres produced with this technique is highly dependent on the type of polymer used.

Spray-Drying

In spray drying techniques, the polymer is dissolved in organic solvent. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting 15, pump setting=10 mL/minute, spray flow=600 Nl/hr, and nozzle diameter=0.5 mm. Microparticles ranging between 1-10 microns in size can be obtained with a morphology which depends on the type of polymer used and the spray drying conditions.

Interfacial Polycondensation

In interfacial polycondensation techniques, one monomer is dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Phase Inversion

Microspheres can be formed from polymers using a phase inversion method wherein a polymer is dissolved in a solvent and the mixture is poured into a strong non solvent for the polymer, to spontaneously produce, under favorable conditions, polymeric microspheres. The method can be used to produce nanoparticles and microparticles in a wide range of sizes, including, for example, about 100 nanometers to about 10 microns. Exemplary polymers which can be used include polyvinylphenol and polylactic acid. In the process, the polymer is dissolved in an organic solvent and then contacted with a non solvent, which causes phase inversion of the dissolved polymer to form small spherical particles, with a narrow size distribution optionally incorporating an antigen or other substance.

Phase Separation

In phase separation, the polymer is dissolved in a solvent to form a polymer solution. While continually stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, forming a particle with a polymeric shell.

Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Hydrogel Particles

Particles made of gel-type polymers, such as alginate and hyaluronic acid, can be produced through traditional ionic gelation techniques. The polymers are first dissolved in an aqueous solution and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The particles are left to incubate in the bath for twenty to thirty minutes in order to allow sufficient time for gelation to occur. Particle size is controlled by using various size extruders or varying either the nitrogen gas or polymer solution flow rates. Chitosan particles can be prepared by dissolving the polymer in acidic solution and crosslinking it with tripolyphosphate. Carboxymethyl cellulose (CMC) particles can be prepared by dissolving the polymer in acid solution and precipitating the particle with lead ions. In the case of negatively charged polymers (e.g., alginate, CMC), positively charged ligands (e.g., polylysine, polyethyleneimine) of different molecular weights can be conically attached.

Other methods known in the art that can be used to prepare particles include, but are not limited to, polyelectrolyte condensation (see Suk et al., Biomaterials, 27, 5143-5150 (2006)); single and double emulsion (probe sonication); particle molding, and electrostatic self-assembly (e.g., polyethylene imine-DNA or liposomes).

Electrospraying or Electrospinning

Electrospraying or electrospinning techniques can be used to prepare particles. In some cases, two or more fluid streams (including liquid jets) are combined together such that the two or more fluid streams contact over spatial dimensions sufficient to form a composite stream. In some cases, there is little or no mixing of the two or more fluid streams within the composite stream. In some variations, the fluid streams are electrically conductive, and in certain cases, a cone-jet may be formed by combining the two or more fluid streams under the influence of an electric field.

In some cases, the composite stream is directed at a substrate, e.g., by the application of a force field such as an electric field. For instance, if the composite stream is charged, an electric field may be used to urge the composite stream towards a substrate. The composite stream may be continuous or discontinuous in some cases, e.g., forming a series of droplets (which may be spherical or non-spherical). In some cases, the composite stream is hardened prior to and/or upon contact with the substrate. For example, the composite stream may be urged towards the substrate under conditions in which at least a portion of the composite stream (e.g., a solvent) is able to evaporate, causing the remaining stream to harden, e.g., to form particles, spheres, rods, or fibers. In some variations, the composite stream fragments in droplets that can lead to particle, sphere, rod, and/or fiber formation.

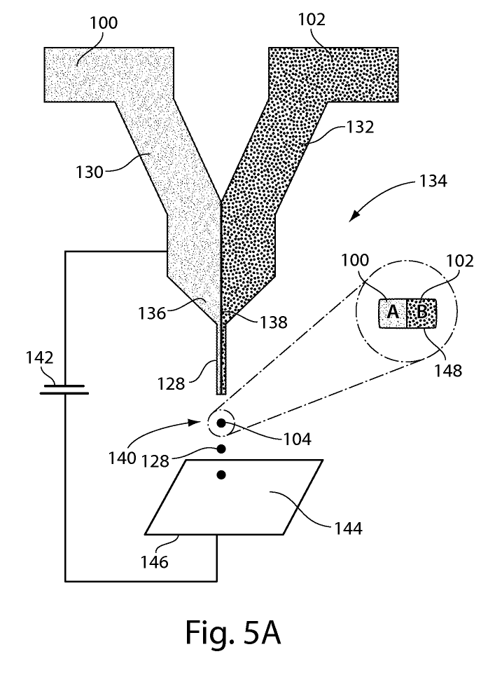

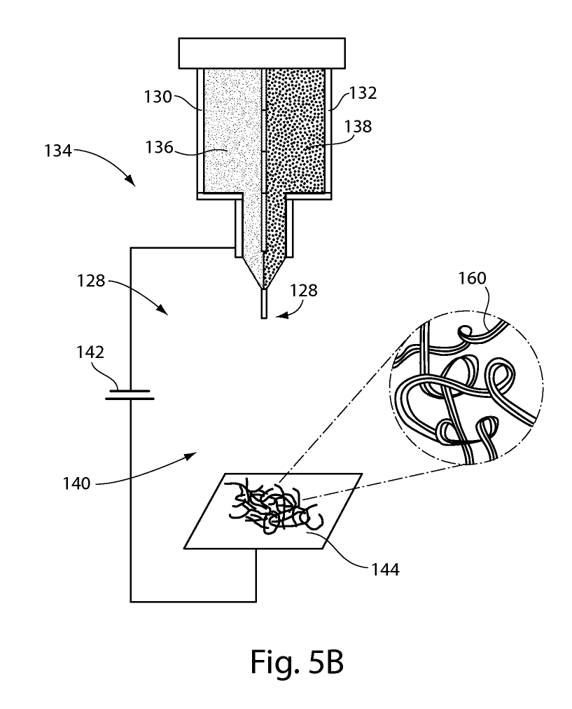

With reference to FIGS. 5A and 5B, schematics illustrating a side-by-side electrojetting apparatus that may be used to form anisotropic particles. FIG. 5A is a schematic of an electrojetting apparatus in which two jetting liquids are combined to form particles. FIG. 5B is a schematic of an electrojetting apparatus in which two jetting liquids are combined to form biphasic fibers. In order to incorporate two different components in each side of the composite stream 128, channels 130, 132 are configured adjacent to each other (i.e., side by side) in nozzle 134. In some variations, channels 130, 132 are capillaries. Channels 130, 132 feed two different jetting liquid streams 136, 138 into region 140 having an electric field generated by power supply 142. Channels 130, 132 are of sufficient dimensions to allow contacting of liquids streams 36, 138 to form composite stream 144. In one variation, this electric field is generated by the potential difference between nozzle 134 and plate 146. Typically, an electric field is formed by applying a potential difference between at least two electrodes from about 0.1 kV to about 25 kV.

It will be appreciated by one skilled in the art that various configurations of plates and geometries may be used to generate the electric field, and therefore are within the scope of the present embodiment. FIG. 5A illustrates one electrospraying variation in which particles 148 are formed. In this variation, ejected composite stream 128 is fragmented due to instabilities thereby forming a spray of droplets. FIG. 5B illustrates one embodiment in which fibers are formed, e.g., when polymer solutions or melts are used as jetting liquids.

### III. Methods of Application and Detection

A. Analytes to be Detected or Measured

1. Normal Physiological Analytes

Blood glucose, insulin, hormone levels are all representative normal analytes to measure, where critical levels trigger a signal. The reactive agents may be used to determine pH (or change pH), temperature (or a change in temperature), and/or the or the presence or absence or the concentration of one or more analytes including, but not limited to,

(a) metal or non-metal ions including, but not limited to, cadmium, calcium, chloride, chromium, copper, iron, magnesium, manganese, molybdenum, phosphorus, potassium, selenium, sodium, sulfur, and zinc;

(b) proteins including, but not limited to, enzymes (proteins having catalytic activity), transport proteins, and structural proteins;

(c) peptides including, but not limited to, C-peptide (as a gauge of insulin production);

(d) amino acids including, but not limited to, naturally occurring, such as alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine, or non-naturally occurring amino acids, such as taurine, citrulline, and ornithine);

(e) nucleic acids including, but not limited to, DNA and RNA;

(f) hormones including, but not limited to, estradiol, estrone, progesterone, progestin, testosterone, androstenedione, follitropin, human chorionic gonadotropin and prolactin;

(g) carbohydrates including, but not limited to, glucose, mannose, galactose, glucosamine, galactoseamine, fucose, amylopectin, amylose, arabinose, fructose, sucrose, etc.;

(h) small molecules, for examples, molecules having a molecular weight less than 1000 Da;

(i) electrolytes including, but not limited to, sodium ion (Na⁺), potassium ion (K⁺⁾, calcium ion (Ca²⁺), magnesium ion (Mg²⁺), chloride ion (Cl⁻), hydrogen phosphate ion (HPO₄²⁻), and hydrogen carbonate ion (HCO₃⁻);

(j) metabolites;

(k) gases (which may be indicative of a disease or disorder of the respiratory tract) including, but not limited to, O₂, CO, CO₂, N₂, and NH₃;

(l) fatty acids including, but not limited to, eicosapentaenoic acid, docosahexanoic acid, linoleic acid, gamma linoleic acid, dihomogamma linoleic acid, and arachidonic acid, as well as the ratio of two or more fatty acids;

(m) lipids including, but not limited to, total cholesterol, high density lipoprotein cholesterol, low density lipoprotein cholesterol, and triglycerides;

(n) cells and/or cell surfaces;

(o) vitamins including, but not limited to, beta-carotene, tocopherols, folic acid, vitamin A, vitamin B1, vitamin B2, vitamin B3, vitamin B6, vitamin C, vitamin D, and vitamin E);

(p) or other analytes of interest.

Examples of analytes to be measured include glucose (e.g., for diabetics); sodium, potassium, chloride, calcium, magnesium, and/or bicarbonate (e.g., to determine dehydration); gases such as carbon dioxide or oxygen; pH; metabolites such as urea, blood urea nitrogen or creatinine; hormones such as estradiol, estrone, progesterone, progestin, testosterone, androstenedione, etc. (e.g., to determine pregnancy, illicit drug use); or cholesterol. Changes in pH can be indicative of one or more disease states.

In the preferred embodiment, these analytes are measured as an “on/off” or “normal/abnormal” situation, where the device indicates a change. The detectable signal on the device may indicate that insulin is needed; a trip to the doctor is needed to check cholesterol; ovulation is occurring; kidney dialysis is needed; drug levels are present (especially in the case of illegal drugs) or the drug levels are too high/too, for example for geriatric patients, particularly in nursing homes; pediatric patients, and medications for which titration is necessary to determine the effective dose, for example, medications to treat mental illness, such as bipolar disorder, depression, schizophrenia, etc.

2. Abnormal Analytes

Examples of abnormal analytes include those indicative of disease, such as cancer specific markers such as CEA and PSA, viral and bacterial antigens, and autoimmune indicators such antibodies to double stranded DNA, indicative of Lupus.

Various pathogens such as bacteria, protozoan parasites (i.e. unicellular eukaryotes) (e.g. Plasmodium) or viruses (e.g. anthrax), and/or markers produced by such pathogens may be detected, for example, by reaction with an antibody directed at a marker produced by a bacteria. Exemplary pathogens include, but are not limited to, viruses (e.g., Adenoviridae, Picornaviridae, Herpesviridae, Hepadnaviridae, Flaviviridae, Retroviridae, Orthomyxoviridae, Paramyxoviridae, Papovaviridae, Rhabdoviridae, Togaviridae), fungi (e.g., molds and yeasts, such as Histoplasma capsulatum, Coccidioides immitis, Candida, and Aspergillus), and/or bacteria (e.g., Mycobacterium tuberculosis, Streptococcus and Pseudomonas, and Shigella, Campylobacter and Salmonella). Pathogens also include parasites. In one embodiment, the organism itself is detected. Alternatively, nucleic acids and/or proteins specific to a particular parasite are detected.

Abnormal analytes also includes drugs, such as, nicotine, prescription drugs, over-the-counter (OTC) drugs, illegal drugs (e.g., cocaine, methamphetamine, LSD, opiates, such as heroin; ecstasy, etc.), anabolic steroids, and prescription drugs prone to abuse. Exemplary prescription drugs prone to abuse include Schedule II, III, IV, and V drugs, such as 1-phenylcyclohexylamine, 1-piperidinocyclohexanecarbonitrile, alfentanil, alphacetylmethadol, alphaprodine, alprazolam, amobarbital, amphetamine, anileridine, apomorphine, aprobarbital, barbital, barbituric acid derivative, bemidone, benzoylecgonine, benzphetamine, betacetylmethadol, betaprodine, bezitramide, bromazepam, buprenorphine, butabarbital, butalbital, butorphanol, camazepam, cathine, chloral, chlordiazepoxide, clobazam, clonazepam, clorazepate, clotiazepam, cloxazolam, cocaine, codeine, chlorphentermine, delorazepam, dexfenfluramine, dextromoramide, dextropropoxyphen, dezocine, diazepam, diethylpropion, difenoxin, dihydrocodeine, dihydromorphine, dioxaphentyl butyrate, dipanone, diphenoxylate, diprenorphine, ecgonine, enadoline, eptazocine, estazolam, ethoheptazine, ethyl loflazepate, ethylmorphine, etorphine, femproponex, fencamfamin, fenfluramine, fentanyl, fludiazepam, flunitrazepam, flurazepam, glutethimide, halazepam, haloxazolam, hexylgon, hydrocodone, hydromorphone, isomethadone, hydrocodone, ketamine, ketazolam, ketobemidone, levanone, levoalphacetylmethadol, levomethadone, levomethadyl acetate, levomethorphan, levorphanol, lofentanil, loperamide, loprazolam, lorazepam, lormetazepam, lysergic acid, lysergic acid amide, mazindol, medazepam, mefenorex, meperidine, meptazinol, metazocine, methadone, methamphetamine, methohexital, methotrimeprazine, methyldihydromorphinone, methylphenidate, methylphenobarbital, metopon, morphine, nabilone, nalbuphine, nalbupine, nalorphine, narceine, nefopam, nicomorphine, nimetazepam, nitrazepam, nordiazepam, normethadone, normorphine, oxazepam, oxazolam, oxycodone, oxymorphone, pentazocine, pentobarbital, phenadoxone, phenazocine, phencyclidine, phendimetrazine, phenmetrazine, pheneridine, piminodine, prodilidine, properidine, propoxyphene, racemethorphan, racemorphan, racemoramide, remifentanil, secobarbital, sufentanil, talbutal, thebaine, thiamylal, thiopental, tramadol, trimeperidine, vinbarbital, allobarbitone, alprazolam, amylobarbitone, aprobarbital, barbital, barbitone, benzphetamine, brallobarbital, bromazepam, brotizolam, buspirone, butalbital, butobarbitone, butorphanol, camazepam, captodiame, carbromal, carfentanil, carpipramine, cathine, chloral, chloral betaine, chloral hydrate, chloralose, chlordiazepoxide, chlorhexadol, chlormethiazole edisylate, chlormezanone, cinolazepam, clobazam, potassium clorazepate, clotiazepam, cloxazolam, cyclobarbitone, delorazepam, dexfenfluramine, diazepam, diethylpropion, difebarbamate, difenoxin, enciprazine, estazolam, ethyl loflazepate, etizolam, febarbamate, fencamfamin, fenfluramine, fenproporex, fluanisone, fludiazepam, flunitraam, flunitrazepam, flurazepam, flutoprazepam, gepirone, glutethimide, halazepam, haloxazolam, hexobarbitone, ibomal, ipsapirone, ketazolam, loprazolam mesylate, lorazepam, lormetazepam, mazindol, mebutamate, medazepam, mefenorex, mephobarbital, meprobamate, metaclazepam, methaqualone, methohexital, methylpentynol, methylphenobarbital, midazolam, milazolam, morphine, nimetazepam, nitrazepam, nordiazepam, oxazepam, oxazolam, paraldehyde, pemoline, pentabarbitone, pentazocine, pentobarbital, phencyclidine, phenobarbital, phendimetrazine, phenmetrazine, phenprobamate, phentermine, phenyacetone, pinazepam, pipradol, prazepam, proxibarbal, quazepam, quinalbaritone, secobarbital, secbutobarbitone, sibutramine, temazepam, tetrazepam, triazolam, triclofos, zalepan, zaleplon, zolazepam, zolpidem, and zopiclone. The analyte to be detected can be the drug itself and/or one or more metabolites of the drug.

Antibodies include, but are not limited to, for example, IgG₄ antibodies associated with food allergies, such as nuts (e.g., almonds, peanuts, cashews, walnuts, etc.), dairy products (e.g., milk, cheese, etc.), meat and poultry, vegetables (e.g., corn); fruits (e.g., melons, oranges, strawberries, tomatoes); shellfish (e.g., crab, shrimp, and/or lobster); eggs; oats; wheat; and legumes; and antibodies that are diagnostic of one or more disease or disorder states (e.g., cancer, autoimmune diseases, etc.;

In the majority of these cases, the detectable signal is an indicator is set as a “warning light”, where the individual is then referred to a physician for further follow-up.

For example, anisotropic particles can be prepared comprising a biocompatible polymer, such as polyethylene oxide (PEO), or polylactic acid (PLA) and/or polyglycolic acid (PGA). The first half of the particles contains a reactive agent that binds to or interacts with a pathogen, such as an antibody to the pathogen and/or a marker produced by the pathogen (e.g., a protein). As a specific example, the pathogen may be anthrax and the antibody may be an antibody to anthrax spores. As another example, the pathogen may be a Plasmodia (some species of which cause malaria) and the antibody may be an antibody that recognizes the Plasmodia. In some cases, these may be soluble molecules that can enter the interstitial fluid. The first half also contains a first colorant, which may be green, e.g., such as fluorescein or GFP. The second half may contain a second colorant, which may be red, e.g., rhodamine.

The particles (or other suitable devices) are suspended in saline and injected into the skin of a human subject. The particles may be injected into the dermis and/or the epidermis, e.g., to form a “mark” within the skin. In the absence of the pathogen, no aggregation of the particles occurs, and the particles are present in a random orientation within the skin; thus, one sees a mixture of red and green (e.g., giving a brown-colored appearance). In the presence of the pathogen (or pathogen marker), however, some aggregation of the particles occurs, such that the particles orient around the pathogen, where the first half of the particles preferentially orients to the pathogen due to the presence of the pathogen reactive partner. Thus, visually, the second colorant will dominate when the particles are aggregated; thus, one sees a brighter red colored appearance compared to the color when the particles are randomly oriented.

3. Other Variables to be Detected or Measured

Other variables that may be detected or measured using the devices described herein include, but are not limited to moisture levels, exposure to elevated levels carbon monoxide, which could be from an external source or due to sleep apnea, too much heat (important in the case of babies whose internal temperature controls are not fully self-regulating) or from fever. Additionally, the devices can be used to measure bacterial levels, or levels of waste products of anaerobic bacteria that may be present in a person's mouth, such as volatile sulfur compounds (e.g. hydrogen sulfide, methyl mercaptan, cadaverine, putrescine, and/or skatole) to determine if the user has elevated levels of compounds and/or bacteria that produce bad breath or is at risk for bad breath.

4. Analysis and Treatment

In addition to determining if one or more analytes are present in an individual, and/or the levels of the analytes in an individual, the devices described herein may also contain one or more therapeutic compounds to treat the disease state, reduce the level of analyte or increase the level of analyte, as needed.

B. Methods of Removing Fluids Containing Analytes to be Detected

In one embodiment, a plurality of particles are administered to the skin or to a mucosal surface by any suitable method or device. Then a fluid to be tested, such as interstitial fluid or blood), is removed from the subject by any suitable means and brought to the site where the particles were administered. Preferably, microneedles are inserted into the skin or mucosal surface to remove the fluid.

In one embodiment, the particles are the devices. In another embodiment, the particles may be embedded in a substrate of a device that is designed to be applied to the skin or mucosal surface (see e.g. FIG. 3B, as an example). In one embodiment, the device is a bandage.

C. Methods of Application

In one embodiment using a one-step diagnostic device, the devices are applied to an individual and then the result is detected based on the site of administration and the device. In general, the devices are administered topically to the skin, injected into the dermis or subcutaneously, or administered to a mucosal surface.

1. Transdermal Surface Administration

The device may be in the form of a bandage, a plastic “watch”, “bracelet” or “ring”, or a specifically designed apparatus for direct application to the skin. The device may be secured physically by restraints or by an adhesive material.

In another embodiment, where the devices are in the form of particles, a plurality of devices may be contained within a cream or a lotion which can be rubbed onto the skin to deliver the devices. In some cases, the device may be administered by a medical practitioner; in other cases, however, the devices may be self-administered.

In some cases, the skin may first be treated with a transdermal penetration enhancer, mechanical abrasion or pressure or ultrasound.

2. Subcutaneous Administration

The devices may be delivered to any location within the skin (or below the skin), e.g., to the epidermis, to the dermis, or subcutaneously, but preferably to the epidermis or subcutaneously to facilitate easily discernible detection. In some cases, a “depot” of devices may be formed within the skin, and the depot may be temporary or permanent. The devices within the depot may eventually degrade or disperse (e.g., if the devices are biodegradable or cleaved at time of reaction), enter the bloodstream, or be sloughed off to the environment.

In one embodiment, the devices may be present in the epidermis and slough off with the epidermis naturally, e.g., on the time scale of days to weeks, depending on the depth of penetration.

In other embodiments, however, an externally applied stimulus is applied to the skin of the subject to at least partially remove and/or inactivate the devices. For instance, light, such as laser light, may be applied to the skin to ablate at least a portion of the skin, including the devices.

In some cases, however, light may be applied to inactivate a portion of the devices (e.g., a reactive agent on the surface of the devices). Many skin ablation lasers may be obtained commercially (for instance, an Er:YAG-laser or a carbon dioxide laser), which are used, for instance, for laser skin resurfacing, facial rejuvenation, ablative removal of skin lesions, or the like. Ablation rates in the skin can be controlled, for instance, by controlling the fluence rate of the laser, the number and/or frequency of pulses (in a pulsed laser), or the like.

In some cases, especially if the devices are colored, the devices after delivery may give the appearance of a “tattoo” or a permanent, or semi-permanent mark within the skin, and the tattoo or other mark may be of any color and/or size. In one embodiment, anisotropic particles, such as those described above that contain one or more reactive agents that are able to bind an analyte, such as glucose, may be delivered by injection into the skin of a subject, and such particles, after deposition within the skin, may react to the presence or absence of the analyte by exhibiting a change in color. The particles may exhibit a color change based on the presence or absence of the analyte, and/or the concentration of the analyte. For instance, the particles may exhibit a first color (e.g., green) when not aggregated, and a second color (e.g., red or brown) when aggregated, or the particles may be invisible when not aggregated, but visible (e.g., exhibiting a color) when aggregated, and thereby form a semi-permanent tattoo.

As just mentioned, the particles may be, for example, anisotropic particles having a first surface region having a first color (e.g., green) and a second surface region having a second color (e.g., red), and the first surface region may contain a reactive partner to an analyte of interest. At low levels of the analyte, the particles may exhibit a combination of the first and second colors, while at higher levels of the analyte, the particles may exhibit more of the second color.

In another embodiment, the color of the particles (or other suitable devices) may be externally controlled with a magnet. This embodiment may be particularly useful for cosmetic applications. Generally, color may be applied to a subject (e.g., in the form of a permanent or a temporary tattoo), and the color may be changed using one or more external magnets. In this embodiment, in addition to having different colors on different parts of the anisotropic particles, a portion of each particle may also contain a magnetically susceptible material, such as iron.

In this example, in the absence of a magnetic field, the particles are present in a random orientation within the skin. However, when a magnetic field is applied, the particles will orient with the magnetic field. Depending on the location of the magnetic field, the particles may become oriented such that the first half of the particles is predominantly visible (leading to a red appearance) or the second half of the particles is predominantly visible (leading to a blue appearance).

The magnetic field may be induced using any suitable technique, for example, with an external device such as a wand, or a bracelet optionally worn by the subject.

a. Hypodermic Needles

A hypodermic needle or similar device may be used to deliver injectable particles, which are suspended in an appropriate carrier, into various tissues. Hypodermic needles are well-known to those of ordinary skill in the art, and can be obtained with a range of needle gauges. Preferred needles are in the 20-30 gauge range. However, in other embodiments, other gauge needles can be used, e.g., 32 gauge, 33 gauge, 34 gauge, etc.

b. Skin Insertion Objects

In one set of embodiments, one or more skin insertion objects may be used to deliver the particles. The skin insertion objects can be constructed to deliver the particles to the dermis and/or to the epidermis, depending on the specific application. The skin insertion objects may be constructed to be inserted into the skin and include a plurality of particles (or other objects). In one embodiment, when the skin insertion objects are inserted into the skin, the particles are released from the skin insertion objects into the skin.

Accordingly, the skin insertion objects may have any suitable shape that allows this to occur, e.g., having the shape of a solid or a hollow needle, which may be cylindrical or may be tapered, etc. For instance, the particles may be fastened to the skin insertion objects with a degree of adhesion such that, when the skin insertion objects are delivered, at least a portion of the particles remain in the dermis and/or epidermis when the skin insertion objects are removed, e.g., due to friction. As another example, a portion of the skin insertion objects may break off upon entry into the skin, thereby delivering the particles. As mentioned, in some cases, one or more skin insertion objects may be present, e.g., immobilized relative to a substrate for simultaneous delivery.

As shown in FIG. 4A, an apparatus (28) containing a plurality of particles (30) adhered to the outer surface (34) of a plurality of solid skin insertion objects (35) may be inserted into the skin by any suitable technique, e.g., manually or by a mechanical apparatus. The plurality of skin insertion objects (35) may be fixed to a substrate (38). As shown FIG. 4B, the skin insertion objects (35) may be hollow. In this embodiment, the particles (30) are delivered into the skin through the hollow portion (36) of the microneedles. As shown FIG. 4C, at least a portion of the skin insertion objects (35) may be constructed to break upon entry into the skin, leaving the particles (30) within the skin.

The skin insertion objects may be formed out of any suitable material, including biocompatible and/or biodegradable materials such as those described herein. In other cases, however, the skin insertion objects are formed from other materials that are not necessarily biocompatible and/or biodegradable.

The skin insertion objects may be delivered to the skin manually, or in some cases, with the aid of a device. The depth of penetration of particles into the skin is determined, at least in part, by the length of the skin insertion objects.

For instance, longer skin insertion objects may be used to penetrate the skin to the level of the dermis, such that at least some of the particles are delivered to the dermis, while shorter skin insertion objects may only penetrate the skin to the level of the epidermis, such that most (if not all) of the particles are delivered into the epidermis.

b. Microneedles

In one embodiment, the skin insertion objects are microneedles. Hollow or solid microneedles may be used to deliver the device to an individual's dermis and/or epidermis. Microneedles such as those disclosed in U.S. Pat. No. 6,334,856, may be used to deliver the devices to the dermis and/or the epidermis, depending on the shape and/or size of the microneedles, as well as the location of delivery. The microneedles may be formed from any suitable material, e.g., metals, ceramics, semiconductors, organics, polymers, and/or composites. Examples include, but are not limited to, pharmaceutical grade stainless steel, gold, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, or polyesters. In some cases, the devices may be delivered via the microneedles; in other eases, however, the microneedles may be first applied to the skin and removed to create passages through the skin (e.g., through the stratum corneum, which is the outermost layer of the skin), then the devices subsequently applied to the skin.

One or more distinct and continuous pathways can be created through the interior of microneedles. In one example, the microneedle has a single annular pathway along the center axis of the microneedle. This pathway can be achieved by initially chemically or physically etching the holes in the material and then etching away microneedles around the hole. Alternatively, the microneedles and their holes can be made simultaneously or holes can be etched into existing microneedles. As another option, a microneedle form or mold can be made, then coated, and then etched away, leaving only the outer coating to form a hollow microneedle. Coatings can be formed either by deposition of a film or by oxidation of the silicon microneedles to a specific thickness, followed by removal of the interior silicon. Also, holes from the backside of the wafer to the underside of the hollow needles can be created using a front-to-backside infrared alignment followed by etching from the backside of the wafer.

One method for hollow needle fabrication is to replace the solid mask used in the formation of solid needles by a mask that includes a solid shape with one or more interior regions of the solid shape removed. One example is a “donut-shaped” mask. Using this type of mask, interior regions of the needle are etched simultaneously with their side walls. Due to lateral etching of the inner side walls of the needle, this may not produce sufficiently sharp walls. In that case, two plasma etches may be used, one to form the outer walls of the microneedle (i.e., a standard etch), and one to form the inner hollow core (which is an extremely anisotropic etch, such as in inductively-coupled-plasma “ICP” etch). For example, the ICP etch can be used to form the interior region of the needle followed by a second photolithography step and a standard etch to form the outer walls of the microneedle.

Alternatively, this structure can be achieved by substituting the chromium mask used for the solid microneedles by a silicon nitride layer on the silicon substrate covered with chromium. Solid microneedles are then etched, the chromium is stripped, and the silicon is oxidized to form a thin layer of silicon dioxide on all exposed silicon surfaces. The silicon nitride layer prevents oxidation at the needle tip. The silicon nitride is then stripped, leaving exposed silicon at the tip of the needle and oxide-covered silicon everywhere else. The needle is then exposed to an ICP plasma which selectively etches the inner sidewalls of the silicon in a highly anisotropic manner to form the interior hole of the needle.

Another example uses the solid silicon needles described previously as “forms” or molds around which the actual needle structures are deposited. After deposition, the forms are etched away, yielding the hollow structures. Silica needles or metal needles can be fowled using different methods. Silica needles can be formed by creating needle structures similar to the ICP needles described above prior to the oxidation described above. The wafers are then oxidized to a controlled thickness, forming a layer on the shaft of the needle form which will eventually become the hollow microneedle. The silicon nitride is then stripped and the silicon core selectively etched away (e.g., in a wet alkaline solution) to form a hollow silica microneedle.

In another example, an array of hollow silicon microtubes is made using deep reactive ion etching combined with a modified black silicon process in a conventional reactive ion etcher. First, arrays of circular holes are patterned through photoresist into SiO₂, such as on a silicon wafer. Then the silicon can be etched using deep reactive ion etching (DRIE) in an inductively coupled plasma (ICP) reactor to etch deep vertical holes. The photoresist is then removed. Next, a second photolithography step patterns the remaining SiO₂ layer into circles concentric to the holes, leaving ring shaped oxide masks surrounding the holes. The photoresist is then removed and the silicon wafer again deep silicon etched, such that the holes are etched completely through the wafer (inside the SiO₂ ring) and simultaneously the silicon is etched around the SiO₂ ring leaving a cylinder.

This latter example can also be varied to produce hollow, tapered microneedles. After an array of holes is fabricated as described above, the photoresist and SiO₂ layers are replaced with conformal DC sputtered chromium rings. The second ICP etch is replaced with a SF₆/O₂ plasma etch in a reactive ion etcher (RIE), which results in positively sloping outer sidewalls.

Metal needles can be formed by physical vapor deposition of appropriate metal layers on solid needle forms, which can be made of silicon using the techniques described above, or which can be formed using other standard mold techniques such as embossing or injection molding. The metals are selectively removed from the tips of the needles using electropolishing techniques, in which an applied anodic potential in an electrolytic solution will cause dissolution of metals more rapidly at sharp points, due to concentration of electric field lines at the sharp points. Once the underlying silicon needle forms have been exposed at the tips, the silicon is selectively etched away to form hollow metallic needle structures. This process could also be used to make hollow needles made from other materials by depositing a material other than metal on the needle forms and following the procedure described above.

nanoBioSciences of Alameda, Calif. that has developed a proprietary drug delivery patch system, dubbed AdminPatch, based on tiny microneedles form pressed out of standard metallic film. The AdminPatch system is an advanced microneedle transdermal delivery technology that painlessly and instantaneously forms hundreds of tiny aqueous channels (‘micropores’) through the stratum corneum and epidermis, the outer resistive surface layers of skin. Proteins and water-soluble molecules can enter the body through these aqueous micropores for either local effect, or by entering the circulation, for systemic effect. The created aqueous channels stay constantly open while AdminPatch is applied on the skin and, therefore, enable the rapid, sustained, and efficient delivery of drugs through these aqueous channels formed in the skin surface. The AdminPatch system is comprised of a single-use disposable AdminPatch and a re-useable handheld Applicator. The disposable AdminPatch contains the proprietary microneedle array laminated on a conventional transdermal drug-in-adhesive patch.

Another disposable adhesive microneedle patch is available from Theraject, Inc., Menlo Park, Calif.

Hollow, porous, or solid microneedles can be provided with longitudinal grooves or other modifications to the exterior surface of the microneedles. Grooves, for example, should be useful in directing the flow of molecules along the outside of microneedles. Polymeric microneedles are also made using microfabricated molds. For example, the epoxy molds can be made as described above and injection molding techniques can be applied to form the microneedles in the molds. In some cases, the polymer is a biodegradable polymer such as those described above.

c. Pressurized Fluids

Pressurized fluids may be used to deliver devices, e.g. particles, for instance, using a jet injector or a “hypospray.” Typically, such apparatuses produce a high-pressure “jet” of liquid or powder (e.g., a biocompatible liquid, such as saline) that drives the devices into the skin, and the depth of penetration may be controlled, for instance, by controlling the pressure of the jet. The pressure may come from any suitable source, e.g., a standard gas cylinder or a gas cartridge. See e.g. U.S. Pat. No. 4,103,684. Pressurization of the liquid may be achieved using compressed air or gas, for instance, by a pressure hose from a large cylinder, or from a built-in gas cartridge or small cylinder.

The depth of penetration of the skin may be controlled by controlling the degree of pressurization of the liquid. In general, higher pressures allow deeper penetration through the skin. Thus, at relatively low pressures, the devices are able to penetrate into the epidermis; at relatively higher pressures, at least some of the devices will penetrate into the dermis of the skin as well.

3. Administration to a Mucosal Surface

The devices are preferably applied to a mucosal surface by spraying a powder, or application of a mucoadhesive device to the tissue. This may be sublingual, buccal, vaginal, rectal, or even intra-nasal.

C. Methods of Detection

The signal can be detected either on the surface or within the device, or in the vicinity of the device.

Devices and uses for devices containing particles are discussed above. These may be used, in some embodiments, to generate a pattern or color which is indicative of the presence and/or amount of analyte. The density, shape, color, or intensity of the pattern or color may provide a yes-no type answer or may be graduated to provide quantitative amounts. This could also be effected by exposure to a pH or temperature change. Optionally, the particles may be exposed to an externally applied force, such as a magnetic field.

The device or skin or tissue surface may change in feel when there is a reaction. For example, shape memory polymers may say “OK” when the cholesterol level is below 150 mg/dl. These may change to read “HIGH” when the cholesterol level exceeds 200 mg/dl. The device may be blank or lack definition at values between these levels.

The device may change taste or smell when reacted with analyte. This may result in a smell such as a food odor being release as a function of a pH or temperature change which released encapsulated scent, or, in the case of a mucosal device, which releases food flavoring such as mint or cinnamon. It is preferred that FDA GRAS ingredients be used as signals.

One embodiment provides a method of determining the presence or amount of analyte that includes administering to the site where analyte is to be measured a single step diagnostic device for determination of the presence and/or amount of an analyte in a subject, wherein the device is administered topically, under or within the skin or mucosal surface, and the device includes: reactive agents which react with an analyte to be detected at the site of administration and agents which generate a signal that can be detected visually, by feel, by smell, or by taste, at the site of reaction with the analyte, unaided by any equipment that may be directly applied to or used by a human with the exception of devices ordinarily used by the individual, such as glasses or a hearing aid. For instance, the determinable change may be a change in appearance (e.g., color), a change in temperature, the production of an odor, etc., which can be determined by a human without the use of any additional equipment.

These devices may be applied to the skin or mucosa to measure a change in temperature indicative of disease or inflammation. In a preferred embodiment, the device would be colorless or a color indicative of normal temperature (for example, green), or the device will display a message such as “OK”. In the event the temperature exceeds a certain level, such as 38° C. (101° F.), the color changes (for example, yellow for caution or red) or the message changes (for example, if shape memory polymers are used) to read “HOT”. These are particularly useful, for example, in a setting such as a day care, where there are a number of babies or young children to supervise, and fevers can occur rapidly.

In another embodiment, the devices may be used to measure a decrease in blood oxygen, or measure the amount of molecules such as glucose, cholesterol, triglycerides, cancer markers, or infectious agents, by providing reactive agents that specifically react with the molecules, and signal generating agents which produce signal in an amount correlated with the amounts of the molecules that react. Alternatively, analogous to the temperature monitor, a present level can be used to create a message that says “C high”, for example, or “insulin!”, for example, which effects a color change.

As discussed above, in another embodiment, the devices may change shape, emit a scent or flavor, or otherwise notify the person of a need to seek further information. In some cases, this might be to seek medical attention where the indicator of a disorder can be confirmed and appropriate medical intervention obtained. In the case of temperature indicative of a fever, the caregiver might measure the temperature using a standard thermometer. In the case of a hormone change, indicative of pregnancy or ovulation, an ELISA test might be performed using a urine sample. In the case of high glucose, this could be confirmed using a standard glucose monitor and a blood sample.

The devices are generally not meant as a final diagnostic, but as an indicator of a condition that requires further follow up.

D. Kits

In another aspect, a kit including one or more of the compositions, e.g., a kit including an anisotropic particle, a kit including a plurality of skin insertion objects, will be prepared. A “kit,” as used herein, typically defines a package or an assembly including one or more of the compositions, for example, as previously described. One or more of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components include, but are not limited to, materials, for example, for using, administering, modifying, assembling, storing, packaging, preparing, mixing, diluting, and/or preserving the compositions components for a particular use, for example, to a sample and/or a subject.

A kit will typically include instructions for preparation and administration, and/or interpretation of the detectable signal. The instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, methods of promoting one or more embodiments discussed herein, for example, methods of promoting the making or use of anisotropic particles or devices containing such particles and/or skin insertion objects, methods of promoting kits as discussed above, or the like. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

Incorporated herein by reference are a U.S. provisional patent application 61/163,733 filed on Mar. 26, 2009, entitled “Determination of Tracers within Subjects”; U.S. provisional patent application 61/163,750 filed on Mar. 26, 2009, entitled “Monitoring of Implants and Other Devices”; and U.S. provisional patent application Ser. No. 61/163,710 filed on Mar. 26, 2009 entitled “Systems And Methods For Creating And Using Suction Blisters or Other Pooled Regions Of Fluid Within The Skin.”

### EXAMPLES

Specific non-limiting examples of devices include, for example, anisotropic particles comprising a biocompatible polymer such as PEO, or a polymer of polylactic acid and/or polyglycolic acid. Such prophetic examples are now described.

In one example, the first half of the particles may contain a glucose binding partner, such as glucose oxidase or glucose 1-dehydrogenase that is able to bind to glucose. The first half also contains a first colorant, which may be green, e.g., such as fluorescein or GFP. The second half may contain a second colorant, which may be red, e.g., rhodamine. Such particles can be suspended in saline and injected into the skin of a human subject. At relatively low levels of glucose, no aggregation of the particles occurs, and the particles are present in a random orientation within the skin; thus, one sees a mixture of red and green (e.g., giving a brown-colored appearance). At relatively high levels of glucose, some aggregation of the particles occurs, such that the particles orient around the glucose, where the first half of the particles preferentially orients to the glucose due to the presence of the glucose binding partner. Thus, visually, the second colorant will dominant when the particles are aggregated; thus, one sees a brighter red colored appearance.

As another example, the first half and the second half may each contain different colorants or dyes (for example, the first half may be red while the second half may be blue). The first half of the particle may also contain a magnetically susceptible material, such as iron, which may be introduced into the fluid stream prior to formation of the particles. In the absence of a magnetic field, the particles are present in a random orientation within the skin. However, when a magnetic field is applied, the particles may become oriented within the magnetic field. The magnetic field may be externally applied. Depending on the position of the magnetic field, the particles may become oriented such that the first half of the particles is predominantly visible (leading to a red appearance) or the second half of the particles is predominantly visible (leading to a blue appearance). The magnetic field may be induced using any suitable technique, for example, with an external apparatus such as a wand, or a bracelet optionally worn by the subject.

As another example, the first half of the particles contains a reactive agent that binds to or interacts with a pathogen. For instance, the reactive agent may be an antibody to the pathogen and/or a marker produced by the pathogen (e.g., a protein). As a specific example, the pathogen may be anthrax and the reactive agent may be an antibody to anthrax spores. As another example, the pathogen may be a Plasmodia (some species of which causes malaria) and the reactive agent may be an antibody that recognizes the Plasmodia. In some cases, these may be soluble molecules that can enter the interstitial fluid. The first half of the particles also contains a first colorant, which may be green, e.g., such as fluorescein or GFP. The second half may contain a second colorant, which may be red, e.g., rhodamine.

As yet another example, the first half of the particles contains a reactive agent that binds to or interacts with a pathogen. For instance, the reactive agent may be an antibody to the pathogen and/or a marker produced by the pathogen (e.g., a protein). As a specific example, the pathogen may be anthrax and the reactive agent may be an antibody to anthrax spores. The first half of the particles also contains a first colorant, which may be green, e.g., such as fluorescein or GFP. The second half may contain a second colorant, which may be red, e.g., rhodamine.

More than one set of anisotropic particles may be used in some cases. For example, in one embodiment, a first set of anisotropic particles contains a first half containing a reactive agent to a species and a second half that contains a first signaling agent, while a second set of anisotropic particles also contains a reactive agent to the species and a second half that contains a second signaling agent. The first and second signaling agents may be, for example, two agents that produce an endothermic or an exothermic reaction when they are brought together, for example, barium hydroxide (Ba(OH)₂) and ammonium nitrate (NH₄NO₃). The first half of the particles also contains as a reactive agent, a glucose binding partner, such as a lectin (e.g., concanavalin A), glucose oxidase or glucose 1-dehydrogenase that is able to bind to glucose. At relatively low levels of glucose, no aggregation of the particles occurs, and no change in temperature is felt by the subject. However, at relatively high levels of glucose, some aggregation of the particles occurs, such that the particles orient around the glucose, where the first halves of the particles preferentially orients to the glucose due to the presence of the glucose binding partner. The second halves of the particles are thus brought into close proximity to each other, allowing the reaction rate between the reactants to increase. In this case, the reaction between barium hydroxide and the ammonium nitrate is an endothermic reaction that yields barium nitrate (Ba(NO₃)₂) and ammonium (NH₃). This may be sensed as a drop in temperature.

In some cases, certain particles described herein can be used as an encoding system. For example, anisotropic particles containing different colorants or dyes may be used, for example, a first half may be substantially transparent while the second half may be blue. The first half of the particle may also contain a magnetically susceptible material, such as iron, which may be introduced into the fluid stream prior to formation of the particles. The particles are suspended in saline and applied into the skin of a subject. The particles may be injected into the dermis and/or the epidermis, e.g., to form a “mark” within the skin. In some cases, the mark will give the appearance of a tattoo. The mark may be used to encode a code word, phrase, or symbol within the subject. The mark may also define an abstract symbol, words, or the like. The mark may also be temporary (e.g., if the particles are delivered primarily to the epidermis) or permanent. In some cases, the mark, once applied to the subject, may be invisible. For example, the particles associated with the mark may include a first half that is colorless and a second half that includes a color, such as red. In the absence of a magnetic field, the particles are present in a random orientation within the skin. Thus, the mark in the skin will appear to be a blend of the first and second colors, and/or the mark in the skin may appear to be similar to the rest of the skin, e.g., if the particles are not present at a relatively high concentration. However, when a magnetic field is applied, the particles may become oriented within the magnetic field, as the first half of the particles contains a magnetically susceptible material. The magnetic field may be externally applied. Depending on the position of the magnetic field, the particles may become oriented such that the second half of the particles is predominantly visible, thereby leading to a colored appearance within the skin. Thus, the particles may be used to encode a secret message that is administered to a subject. As the particles are relatively transparent, they may be difficult or impossible for another person to find without knowing the location and nature of the encoded information. However, exposure of the subject to a magnetic field having suitable intensity may cause the particles to become aligned, which could be determined as an encoded signal.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

1. A single step diagnostic device for determination of the presence and/or amount of an analyte in a subject, wherein
the device is in a form suitable for administration topically, under or within the skin or mucosal surface,
and wherein the device comprises
a reactive agent that reacts with or interacts with an analyte to be detected at the site of administration, and
a signaling agent which, alone and/or in combination with another species, generates a signal that can be detected visually, by feel, by smell, or by taste, at the site of reaction with the analyte, wherein the reactive agent and the signaling agent may be the same or different.

2-16. (canceled)

17. A method of determining the presence or amount of analyte comprising administering to a subject topically, under or within the skin or mucosal surface to the site where analyte is to be measured
a single step diagnostic device for determination of the presence and/or amount of an analyte in the subject, wherein
the device comprises
a reactive agent that reacts with an analyte to be detected at the site of administration and
a signaling agent, which, alone and/or in combination with another species, generates a signal that can be detected visually, by feel, by smell, or by taste, at the site of reaction with the analyte, without reference to an external or secondary device or reference sample.

18-22. (canceled)

23. A method of determining a target indicative of an analyte, comprising:
exposing the target to a group of particles, at least some particles of the group of particles having at least two distinct surface regions including at least a first surface region and a second surface region, wherein the first surface region is able to fasten the target;
fastening the first surface region of the at least some particles to the analyte to cause a change in particle orientation; and
determining a determinable feature of the particles, thereby determining the target, to determine the analyte.

24-70. (canceled)

71. A method, comprising:
delivering particles, suitable for determining an analyte within the skin of a subject for a period of time of at least about a 15 minutes to the skin of the subject via a plurality of skin insertion objects.

72-82. (canceled)

83. A method, comprising:
administering, into the skin of a subject, particles having at least two distinct regions, each region being present on the surface of the particles.

84-85. (canceled)

86. A diagnostic sensor composition foreign to a subject, constructed to be resident in the epidermis of the subject to an extent greater than in the dermis of the subject, the composition responsive to an analyte so as to produce a detectable signal in the presence of the analyte distinguishable from a signal in the absence of the analyte.

87-92. (canceled)

93. A method, comprising:
determining a physical condition of a subject by determining the visual appearance of a material located in the skin of the subject.

94-97. (canceled)

98. A composition, comprising:
a temporary tattoo positioned primarily within the epidermis.

99-101. (canceled)

102. A temporary, fully integrated continuous sensor on or in the skin that provides a sensory signal determinable by a user without the aid of external equipment.

103. (canceled)
